(19) United States
(12) Patent Application Publication
(10) Pub. No.: US 2025/0263694 A1
(43) Pub. Date: Aug. 21, 2025

(54) CONJUGATED COMPOSITION AND METHOD FOR DELIVERING A THERAPEUTIC NUCLEIC ACID SEQUENCE TO A LIVING CELL

(71) Applicant: Twister Biotech, Inc., Houston, TX (US)

(72) Inventors: Yuming XIE, Sugar Land, TX (US); Michael PAVIA, Houston, TX (US); James Daniel CATANESE, Jr., Houston, TX (US); Christopher COKER, Houston, TX (US)

(73) Assignee: Twister Biotech, Inc., Houston, TX (US)

(21) Appl. No.: 18/864,920

(22) PCT Filed: May 13, 2023
(86) PCT No.: PCT/US2023/022161

§ 371 Date: Nov. 12, 2024

Related U.S. Application Data
(60) Provisional application No. 63/341,458, filed on May 13, 2022.

(51) Int. Cl. C12N 15/11

(52) U.S. Cl. CPC C12N 15/11; C12N 2310/351; C12N 2310/532

(57) ABSTRACT

A conjugated composition and methods of using the same for treatment of a living cell, are provided. The conjugated composition includes a nucleic acid sequence directly or indirectly conjugated to at least one N-Acetylgalactosamine (GalNAc) molecule, or to a derivative of a GalNAc molecule. The conjugation is provided by a linker. The conjugated composition is structured to enable the at least one GalNAc molecule or derivative to deliver the nucleic acid sequence into a cell, for example, into a liver cell. The nucleic acid sequence can be a therapeutic supercoiled DNA minicircle that has a therapeutic gene for delivery to the cell.

### CROSS-REFERENCE TO RELATED APPLICATION

This application claims benefit of priority of U.S. provisional application No. 63/341,458, filed May 13, 2022, which is hereby incorporated by reference in its entirety.

### FIELD OF THE INVENTION

The present invention relates to a conjugated composition and to the delivery of a therapeutic nucleic acid sequence to a living cell.

### BACKGROUND OF THE INVENTION

Current methods and compositions for delivering nucleic acid sequences into cells suffer from numerous limitations, including suboptimal particle size, suboptimal nucleic acid cargo protection from digestive agents, non-targeted delivery, inadequate endosomal escape, toxicity, non-biodegradability, and poor transfection efficiency. The use of N-Acetylgalactosamine (GalNAc) has been shown to aid in the delivery of nucleic acid sequences to cells. In particular, Debacker et al. (“Delivery of Oligonucleotides to the Liver with GalNAc: From Research to Registered Therapeutic Drug” Molecular Therapy, 2020, Vol. 28, No. 8) shows the targeted delivery of oligonucleotides to liver hepatocytes using GalNAc molecules. The oligonucleotides that are delivered to liver cells in Debacker et al., however, are limited to small interfering RNAs (siRNAs).

A need exists for a new method and composition for delivering therapeutic deoxyribonucleic acid (DNA) sequences into cells, for example, therapeutic DNA sequences that can metabolize in a living cell to treat the cell.

### SUMMARY OF THE INVENTION

A feature of the present invention is a conjugated composition that can effectively deliver a therapeutic nucleic acid sequence to a living cell.

Another feature of the present invention is to provide a method for effectively delivering a therapeutic nucleic acid sequence to a living cell to treat the living cell.

Another feature of the present invention is to provide a method of making a conjugated composition that can deliver a therapeutic nucleic acid sequence to a living cell.

Additional features and advantages of the present invention will be set forth in part in the description that follows, and in part will be apparent from the description, or may be learned by practice of the present invention. The objectives and other advantages of the present invention will be realized and attained by means of the elements and combinations particularly pointed out in the description and appended claims.

To achieve these and other advantages, and in accordance with the purposes of the present invention, as embodied and broadly described herein, the present invention relates to a conjugated composition comprising a therapeutic nucleic acid sequence directly or indirectly conjugated to at least one N-Acetylgalactosamine (GalNAc) molecule or at least one derivative of a GalNAc molecule. The direct or indirect conjugation can be provided by a linker. The therapeutic nucleic acid sequence can be a circular nucleic acid (i.e., closed loop). The conjugated composition is structured to enable the at least one GalNAc molecule to deliver the therapeutic nucleic acid sequence to a cell.

While reference is made herein to “at least one GalNAc molecule,” it is to be understood that such reference can also refer to at least one derivative of a GalNAc molecule. Exemplary derivatives of a GalNAc molecule that can be used according to the present invention include derivatives wherein one or more of the hydroxyl groups of the GalNAc molecule are replaced or substituted with a different substituent, for example, an organic group, a hydrocarbon, or an acetyl group. One such derivative is a tri-acetyl-substituted GalNAc molecule, although mono-acetyl-substituted, di-acetyl-substituted, or other substituted GalNAc molecules can also be used.

The present invention further relates to a method of treating a living cell. The method includes the step of contacting a living cell with a conjugated composition as described herein. The conjugated composition can comprise, for example, a therapeutic supercoiled DNA minicircle having from 100 base pairs to 2.5 kilo base pairs. The therapeutic supercoiled DNA minicircle is directly or indirectly conjugated to at least one GalNAc molecule or to at least one derivative of a GalNAc molecule, by a linker. The at least one GalNAc molecule of the conjugated composition can bind to a receptor of the living cell. The conjugated composition is taken up in an endosome of the living cell where the conjugated composition dissociates from the receptor. The at least one GalNAc molecule is then cleaved from the therapeutic supercoiled DNA minicircle, at the linker. Once inside the cell, the therapeutic supercoiled DNA minicircle can then be imported into the nucleus of the cell to treat that cell.

The present invention further relates to a treated cell in combination with a remnant GalNAc molecule that includes a remnant of a linker. The treated cell can comprise a human liver cell and a therapeutic supercoiled DNA minicircle within the human liver cell. The remnant GalNAc molecule including a remnant of the linker results from cleaving a conjugated composition that originally included the therapeutic supercoiled DNA minicircle conjugated to at least one GalNAc molecule. It is to be understood that the remnant GalNAc molecule can be a remnant GalNAc derivative molecule and that the at least one GalNAc molecule can be at least one GalNAc derivative molecule. The therapeutic supercoiled DNA minicircle in the treated cell results from cleavage of a conjugated composition.

The conjugated composition can include a linker. The linker can include, for example, an ether bond, an ether bridge, an oxygen bond, or an oxygen bridge. One or more remnants of the linker results from the cleavage. A remnant of the linker resulting from the cleavage can remain attached to the GalNAc molecule. A remnant of the linker resulting from the cleavage can remain attached to the therapeutic supercoiled DNA minicircle. A first remnant of the linker can remain attached to the GalNAc molecule while a second remnant of the linker can remain attached to the therapeutic supercoiled DNA minicircle.

It is to be understood that both the foregoing general description and the following detailed description are exemplary and explanatory only and intended to provide a further explanation of the present invention, as claimed.

The accompanying drawings, which are incorporated in and constitute a part of this application, illustrate some of the features of the present invention and together with the description, serve to explain the principles of the present invention.

### BRIEF DESCRIPTION OF THE DRAWINGS

FIG. 1 is a schematic illustration of a chemical structure of a conjugated composition including three tri-acetyl-substituted GalNAc molecules conjugated with a supercoiled DNA minicircle, by a linker, the linker and supercoiled DNA minicircle together forming a DNA triplex.

FIG. 2 is a schematic illustration of the chemical structure of a conjugated composition including three tri-acetyl-substituted GalNAc molecules conjugated with a supercoiled DNA minicircle, by a linker.

FIG. 3 is a chart showing cell viability of a cell line of A549 when treated with LPEI, BPEI, SP, SPG, and LPN at different concentrations.

FIG. 4 is a chart showing cell viability of a cell line of Ovcar8 when treated with LPEI, BPEI, SP, SPG, and LPN at different concentrations.

FIG. 5 is a chart showing cell viability of a cell line of OV90 when treated with LPEI, BPEI, SP, SPG, and LPN at different concentrations.

FIG. 6 is a chart showing relative growth of a human liver cancer cell line (HepG2) when treated with a control, star polymer GalNAC, and minivectors coated with star polymer GalNACs.

### DETAILED DESCRIPTION OF THE PRESENT INVENTION

The present invention, in part, relates to a conjugated composition including N-Acetylgalactosamine (GalNAc), or a derivative thereof, conjugated with a therapeutic nucleic acid sequence. The present invention also relates, in part, to a method of using the conjugated composition for delivering the therapeutic nucleic acid sequence to a living cell.

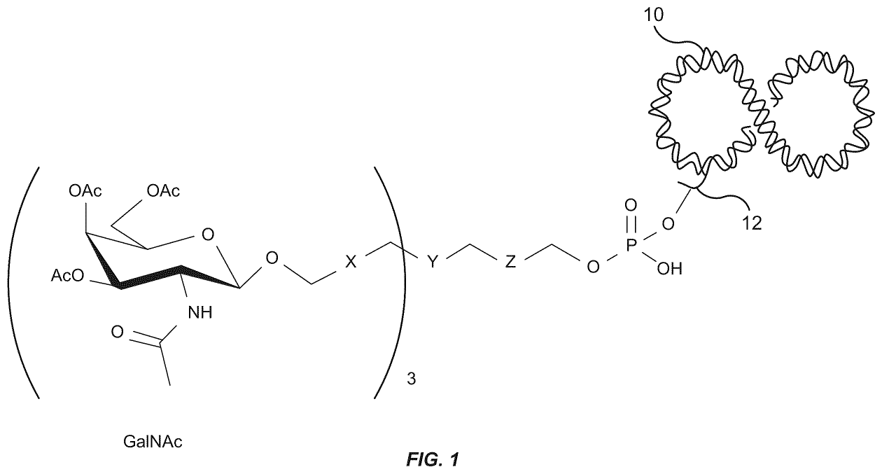

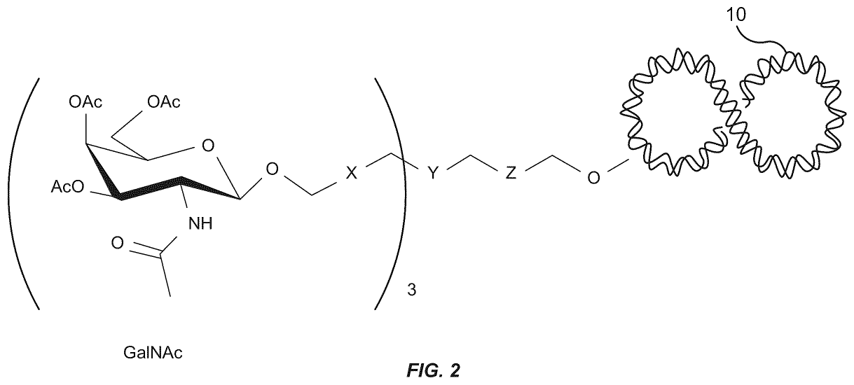

The conjugated composition includes a nucleic acid sequence directly or indirectly conjugated to at least one GalNAc molecule, or to at least one derivative of a GalNAc molecule. The conjugation is made through a linker. While reference is made herein to “at least one GalNAc molecule,” it is to be understood that such reference can also refer to at least one derivative of a GalNAc molecule. Exemplary derivatives of a GalNAc molecule that can be used according to the present invention include derivatives wherein one or more of the hydroxyl groups of the GalNAc molecule are replaced or substituted with a different substituent, for example, an organic group, a hydrocarbon, or an acetyl group. One such derivative is a tri-acetyl-substituted GalNAc molecule as shown, for example, in FIGS. 1 and 2. Although labelled “GalNAc” in FIGS. 1 and 2, it is to be understood that the structures so-labelled are actually derivatives of a GalNAc molecule, specifically, a tri-acetyl-substituted GalNAc molecule. Mono-acetyl-substituted, di-acetyl-substituted, or other substituted GalNAc molecules can also be used.

The conjugated composition is structured to enable the at least one GalNAc molecule to deliver the nucleic acid sequence into a cell, for example, into a human liver cell. The liver cell can be a hepatocyte.

GalNAc is an amino sugar derivative of galactose. The GalNAc molecule binds with liver hepatocytes. Specifically, liver hepatocytes express asialoglycoprotein receptors (ASGPR) that bind and clear circulating glycoproteins from which a sialic acid residue has been removed to expose sugar residues. ASGPR are high-capacity, rapidly internalizing receptors and number approximately 500,000 copies per hepatocyte. The at least one GalNAc molecule of the conjugated composition binds to the ASGPR and is taken up in endosomes, wherein the conjugate composition dissociates from the receptor. Then, the at least one GalNAc molecule is cleaved from the therapeutic nucleic acid sequence before the therapeutic nucleic acid sequence is released into the cell's cytoplasm. From there, the DNA can enter the nucleus to exert its therapeutic effect.

The use of GalNAc molecules to deliver small oligonucleotides to the liver is described in detail, for example, in Debacker et al., “Delivery of Oligonucleotides to the Liver with GalNAc: From Research to Registered Therapeutic Drug,” Molecular Therapy, 2020, Vol. 28, No. 8, and described in Nair et al., “Multivalent N-Acetylgalactosamine-Conjugated siRNA Localizes in Hepatocytes and Elicits Robust RNAi-Mediated Gene Silencing,” J. Am. Chem. Soc. 2014, 136, pp. 16958-16961, both of which are incorporated herein by reference in their entireties.

The ASGPR is formed of two subunits, ASGR1 and ASGR2, assembled in a hetero-oligomer at different ratios. The avidity of the receptor is dependent on the number of ligands attached to the receptor. According to the present invention, it has been found that the affinity of the ASGPR for a trimer of GalNAc is 1,000-fold higher than for a dimer, and 1,000-fold higher than for a monomer. It has also been found that a tetramer has just a slightly higher affinity for the receptor than a trimer. The conjugated composition of the present invention can include one GalNAc molecule, two GalNAc molecules, three GalNAc molecules, four GalNAc molecules, or more, for example, a trivalent cluster of GalNAc molecules (three GalNAc molecules).

With a trivalent cluster, the distance between the GalNAc molecules can be from 10 Å to 40 Å, from 15 Å to 35 Å, from 20 Å to 30 Å, or 25 Å.

The conjugated composition of the present invention can be used for the treatment of cells, such as damaged cells, abnormal cells, or diseased cells. The conjugated composition is delivered in vitro or in vivo to cells in need of treatment. The cells can be cancerous cells, malignant cells, or other types of diseased cells. The treatment can be used for diseases of the liver, including liver cancer, liver fibrosis, and others.

The conjugated composition can be used in an effective amount. As used herein, the term “effective amount” refers to an amount which, when administered in a proper dosing regimen, is sufficient to treat (therapeutically or prophylactically) the target disorder. For example, an effective amount is sufficient to reduce or ameliorate the severity, duration, or progression of the disorder being treated, prevent the advancement of the disorder being treated, cause the regression of the disorder being treated, and/or enhance or improve the prophylactic or therapeutic effect(s) of another therapy. The amount of conjugated composition can be dosed at least from 5 micrograms up to 5000 micrograms, from 50 micrograms up to 3000 micrograms, from 100 micrograms up to 1000 micrograms, from 200 micrograms up to 800 micrograms, or from about 400 micrograms up to 600 micrograms, per day.

In certain embodiments, the conjugated composition is used to treat patients having a liver disease. For example, the conjugated composition can be used for the treatment of hepatitis B, genetic diseases, non-alcoholic fatty liver disease, and the like. Other treatments can take advantage of the liver as a secretory organ, aiming to treat diseases that do not primarily affect the liver by modulation proteins secreted by hepatocytes. The conjugated composition can be used to treat any blood related ailments, such as, but not limited to, diabetes, blood cancers, cardiovascular disease, high blood pressure, and the like.

The therapeutic nucleic acid sequence can be a synthetic nucleic acid sequence. Although deoxyribonucleic acid sequences are described often herein, the therapeutic nucleic acid sequence can instead or additionally include at least one of DNA, shRNA, RNA, siRNA, miRNA, analogues thereof, and combinations thereof. In some embodiments, the nucleic acid sequence includes nucleic acid analogues, such as morpholinos modified nucleic acids and thiophosphate modified nucleic acids. In more specific embodiments, the nucleic acids can include plasmid DNA.

In some embodiments, the nucleic acid sequence can include a gene. In some embodiments, the gene can include but is not limited to, GFP, luciferase, beta-GAL, VEGF, HER-2, SRC-3, HIF-1, Tp53, FOXM1, or a combinations thereof. The Tp53 can be a tetrameric p53 that binds DNA 1,000 fold more than a monomeric wild type p53. Dominant negative tetrameric p53 occurs when a mutant monomeric p53 (e.g., cancerous) is bound up in the achieved tetramer thereby rendering it quiescent or harmful (“poisoned”). Tp53 is a modified p53 gene cassette that can be encoded on nucleic acid structures (e.g., DNA). When expressed, Tp53 is active during each stage of the cell cycle (i.e., it is constitutive). Further, monomeric Tp53 only forms a tetramer with other monomeric Tp53 thereby obviating the “dominant negative” problem associated with wild type p53.

The nucleic acid sequence can be a linear, capped, close-ended, closed-looped, or of a circular shape. The nucleic acid sequence can be single, double, or triple-stranded DNA.

Circular nucleic acids (CNAs) are nucleic acid molecules with a closed-loop structure. The closed-loop structure includes the two ends of the nucleic acid joined together. This feature comes with a number of advantages including resistance to nuclease degradation, much better thermodynamic stability, and the capability of being replicated by a DNA polymerase in a rolling circle manner.

In certain embodiments, the circular nucleic acid sequence is a minivector. Minivector is a name applicant attributes to the Baylor College of Medicine, Houston, Texas. An exemplary useful minivector that can be used according to the present invention is a supercoiled DNA minicircle. The supercoiled DNA minicircle can be a supercoiled DNA minicircle as described in U.S. Pat. No. 7,622,252 B2, a “MiniVector” as described in U.S. Pat. No. 8,729,044 B2, a “MiniVector” as described in U.S. Pat. No. 8,460,924 B2, or a “minivector” as described in U.S. Pat. No. 9,267,150 B2, all of which are incorporated herein by reference in their entireties.

Naked smaller looped DNA structures (i.e., those with less than ˜4,000 base pairs) exhibit substantially improved survival in serum as compared to larger naked looped DNA structures, as described in Zhao et al., “Transfection of shRNA-encoding Minivector DNA of a few hundred base pairs to regulate gene expression in lymphoma cells,” Gene Therapy, (2011) 18, pp. 220-224, which is incorporated herein by reference in its entirety. While protection from nucleases and other digestive particles is desirable, it may be preferable for other reasons (e.g., improved ability to penetrate smaller organ fenestrations) to deliver naked DNA particles on a targeted basis to the liver. Small, naked circular looped DNA particles conjugated to GalNac molecules afford this possibility.

The supercoiled DNA minicircle can be a double-stranded, closed-end, circular, ultrapure, supercoiled DNA nanoparticle that is absent or free of a selection marker (e.g., antibiotic resistance gene), absent or free of a bacterial origin of replication, or absent or free of both. The lack of a selection marker and of a bacterial origin of replication allows for persistent transgene and therapeutic sequence expression with reduced silencing and immune responses associated with plasmid DNA vectors.

The definition of ultrapure DNA is a DNA that is free of unrecombined parent plasmid, mini plasmid bioproduct, and other unwanted bacterial sequences. The DNA can contain more than 95% DNA, more than 96% DNA, more than 97% DNA, more than 98% DNA, more than 99% DNA, or more than 99.9% DNA relative to the unrecombined parent plasmid, the mini plasmid bioproduct, and the other unwanted bacterial sequences.

DNA supercoiling refers to the amount of twist and writhe in a particular DNA strand, which determines the amount of torsional strain on it. A given strand can be “positively supercoiled” or “negatively supercoiled” (more or less tightly wound relative to relaxed DNA, respectively). In a “relaxed” double-helical segment of B-DNA, the two strands twist around the helical axis once every 10.4-10.5 base pairs of the sequence. Adding or subtracting twist and/or writhe imposes strain. If a DNA segment under torsional strain is closed into a circle by joining its two ends, and then allowed to move freely, the supercoiling is redistributed at some proportion of twist and writhe depending on the amount of torsional strain and length of the circle. The level of supercoiling cannot be changed unless at least one of the strands of DNA is broken.

Lkₒ is the number of turns in a relaxed DNA molecule, for example, in a B type, DNA molecule. Lkₒ is determined by dividing the total base pairs of the molecule by the ratio of base pairs to turns (bp/turn). Depending on the reference, the number of base pairs to turns (bp/turn) might be 10.4, 10.5, or 10.6. So, for example, the Lkₒ of a relaxed B type, DNA molecule can be determined by using the reference 10.4 and the total number of base pairs of the DNA molecule.

Using the above example, for a relaxed B type, DNA molecule having 416 base pairs, the Lkₒ would be determined by dividing 416 base pairs (bp) by 10.4 base pairs per turn (bp/turn). As a result, the number of turns (Lkₒ) of the DNA molecule would equal 40.

DNA supercoiling can be described numerically by changes in the linking number Lk. The linking number is a descriptive property of supercoiled DNA. Lk is the number of crosses a single strand makes across the other, often visualized as the number of Watson-Crick twists found in circular DNA in a (usually imaginary) planar projection. This number is physically “locked in” at the moment of covalent closure of the circular DNA, and cannot be altered without strand breakage.

Tw, called “twist,” is the number of Watson-Crick twists in the DNA molecule when it is not constrained to lie in a plane. Native DNA (i.e., DNA isolated from a cell) is usually found to be negatively supercoiled (i.e., underwound). If one goes around the superhelically twisted DNA molecule, counting secondary Watson-Crick twists, that number is different from the number counted when the DNA molecule is constrained to lie flat. In general, the number of secondary twists in the native DNA is expected to be the “normal” Watson-Crick winding number, meaning a single 10-base-pair helical twist for every 34 Å of DNA length.

Wr, called “writhe,” is the number of superhelical twists. Since biological circular DNA is usually underwound, Lk is generally less than Tw, which means that Wr is typically a negative.

If DNA is underwound, it is under strain, exactly as a metal spring is strained when forcefully unwound. The appearance of supertwists will allow the DNA to relieve its strain by taking on negative supertwists, which correct the secondary underwinding in accordance with the topology equation below.

The topology of the DNA is described by the equation Lk=Tw+Wr, in which the linking number is equivalent to the sum of Tw, that is the number of twists or turns of the double helix, and Wr, that is the number of coils or “writhes.” If there is a closed DNA molecule, the sum of Tw and Wr, or the linking number, does not change, hence, Lk=Tw+Wr. There may, however, be complementary changes in Tw and Wr without changing their sum.

The topology equation shows that there is a one-to-one relationship between changes in Tw and Wr. For example, if a secondary “Watson-Crick” twist is removed, then a right-handed supertwist must have been removed simultaneously. Alternatively, if the DNA is relaxed, with no supertwists, then a left-handed supertwist is added.

The change in the linking number, ΔLk, is the actual number of turns in the DNA molecule, Lk, minus the number of turns in the relaxed DNA molecule Lkₒ:

$$Δ Lk = Lk - {Lk}_{o}$$

If the DNA is negatively supercoiled, then ΔLk<0. The negative supercoiling implies that the DNA is underwound.

A standard expression independent of the molecule size is the “specific linking difference” or “superhelical density,” denoted σ, which represents the number of turns added or removed relative to the total number of turns in the relaxed DNA molecule, which is indicative of the level of supercoiling. Accordingly, the equation for “specific linking difference” or “superhelical density” is

$$σ = Δ Lk / {Lk}_{o}$$

The Gibbs free energy associated with the coiling is given by the equation below

$$ΔG / N = 10 RT {σ}^{2}$$

The difference in Gibbs free energy between the supercoiled circular DNA and uncoiled circular (or relaxed) DNA, with N>2000 bp, is approximated by:

$$ΔG / N = 700 Kcal / bp * ( Δ Lk / N )$$

or, 16 cal/bp.

The linking number Lk of supercoiled DNA is the number of times the two strands are intertwined (and both strands remain covalently intact). Thus, Lk cannot change. The reference state (or parameter) Lk₀ of a circular DNA duplex is its relaxed state. In this state, its writhe W=0. Since Lk=Tw+Wr, in a relaxed state Tw=Lk. Thus, for a 400 bp relaxed circular DNA duplex, Lk˜40 (assuming ˜10 bp per turn in B-DNA). Thus, Tw is also ˜40.

Given that Lk=Tw+Wr, two examples of positive supercoiling are:

$$Tw = + 3 , Wr = 0 , then Lk = + 3$$

$$Tw = + 2 , Wr = + 1 , then Lk = + 3$$

Given that Lk=Tw+Wr, two examples of negative supercoiling are:

$$Tw = - 3 , Wr = 0 , then Lk = - 3$$

$$Tw = - 2 , Wr = - 1 , then Lk = - 3$$

An exemplary supercoiled DNA minicircle according to the present invention, is smaller than a typical DNA strand. For example, the supercoiled DNA minicircle can be sized from 100 base pairs (bp) to 8 kilo base pairs (kbp), from 250 bp to 5 kbp, from 300 bp to 2.5 kbp, from 400 bp to 2 kbp, from 500 bp to 1.8 kbp, from 600 bp to 1.7 kbp, from 700 bp to 1.6 kbp, from 800 bp to 1.5 kbp, from 900 bp to 1.4 kbp, from 1 kb to 1.3 kbp, or from 1.1 kb to 1.2 kbp. The supercoiled DNA minicircles can be made in size increments of about 100 bp or fewer. Because of their small size, supercoiled DNA minicircles are transfected with high efficiency.

The supercoiled DNA minicircle can have an outer width of from 1 nm to 500 nm, from 2 nm to 300 nm, from 5 nm to 100 nm, from 10 nm to 80 nm, from 20 nm to 60 nm, or from 30 nm to 40 nm, such as from 2 nm to 80 nm. The outer width is measured perpendicular to a length of the supercoiled DNA and is the widest dimension of the supercoiled DNA minicircle.

The supercoiled DNA minicircle can have a length of from 10 nm to 1000 nm, from 15 nm to 800 nm, from 20 nm to 600 nm, from 30 nm to 500 nm, from 40 nm to 200 nm, from 50 nm to 150 nm, from 60 nm to 100 nm, from 70 nm to 90 nm, or 80 nm, such as from 20 nm to 300 nm. The length of the supercoiled DNA is the longest dimension of the supercoiled DNA minicircle.

The supercoiled DNA minicircle can be engineered to contain a small gene and promoter yet still maintain a small size (less than, for example, about 2000 base pairs, much smaller than any plasmid bearing a functional gene for gene therapy). The supercoiled DNA minicircle delivers small genes that are transcribed and translated into functional proteins.

The supercoiled DNA minicircle can include a nucleic acid molecule with merely an expression cassette, that is, with merely a promoter and a nucleic acid sequence, wherein the nucleic acid sequence can be, for example, a sequence encoding for shRNA targeted to a specific mRNA transcript. In another embodiment, the expression cassette can include merely a promoter and a nucleic acid sequence, wherein the nucleic acid sequence includes a gene encoding for expression of a specific protein. Importantly, the expression cassette contains minimal or no bacterial-originated sequences.

The supercoiled DNA minicircle can be labeled, e.g., using a chemical moiety, as desired. Representative labels include fluorescein, biotin, cholesterol, dyes, modified bases and modified backbones. Representative dyes include: 6-carboxyfluorescein, 5-/6-carboxyrhodamine, 5-/6-Carboxytetramethylrhodamine, 6-Carboxy-2′-, 4-, 4′-, 5′-, 7-, 7′-hexachlorofluorescein), 6-Carboxy-2′-, 4-, 7-, 7′-tetrachlorofluorescein), 6-Carboxy-4′-, 5′-dichloro-2′-, 7′-dimethoxyfluorescein, 7-amino-4-methylcoumarin-3-acetic acid), Cascade Blue, Marina Blue, Pacific Blue, Cy3, Cy5, Cy3.5, Cy5.5, IRDye700, IRDye800, BODIPY dye, Texas Red, Oregon Green, Rhodamine Red, Rhodamine Green. Additional modifications can also include modified bases (e.g., 2-aminopurine, methylated bases), or modified backbones (e.g., phosphorothioates, where one of the non-bridging oxygens is substituted by a sulfur; 2′-O-methyl-RNA-oligonucleotides; methyl-phosphate oligonucleotides). Multiple labels, including chemical moieties and/or modified bases and/or modified backbones, can be used simultaneously, if desired. Methods of labeling nucleotides are described, for example, in Robert E. Farrell, “Nucleic Acid Probe Technology,” RNA Methodologies (Third Edition), 2005, pp. 285-316, and Tabor et al., “Enzymatic Labeling of Nucleic Acids,” Current Protocols in Immunology, May 2001, Chapter 10, Unit 10.10, both of which are incorporated herein by reference in their entiretics.

The present invention can utilize multiple types of supercoiled DNA minicircles in a single conjugated composition, as well as supercoiled DNA minicircles with multiple targets. For example, two or more separate supercoiled DNA minicircles can be designed, in which each individual supercoiled DNA minicircle encodes a different nucleic acid sequence that comprises a portion or a domain of a single protein, such that when the individual supercoiled DNA minicircles are expressed in a single cell, the portions or domains come together to form a single active protein. In addition, polymer forms of supercoiled DNA minicircles can also be formed during in vivo recombination. Moreover, the present invention encompasses the use of sequences encoding multiple therapeutic shRNAs and supercoiled DNA minicircles with simultaneous multi-gene targeting in transfected cells, resulting in a highly sensitive and specific gene therapy.

The nucleic acid sequence is conjugated to at least one GalNAc molecule by a linker. The linker can be covalently bonded, non-covalently bonded, or a combination thereof to the nucleic acid sequence and the at least one GalNAc molecule. Examples of non-covalent bonds include ionic interactions, acid-base interactions, hydrogen bonding interactions, pi-stacking interactions, van der Waals interactions, adsorption, physisorption, self-assembly, sequestration, hydrophobic interactions, and combinations thereof. The linker can be a cleavable linker or a stable linker. The linker can be a linear molecule such as hydrocarbon chains, ethers, esters, and a combination thereof. In certain embodiments, a phosphodiester can create the link between the nucleic acid sequence and the at least one GalNAc molecule. Other linkers can include peptides, oligosaccharides, lipid chains, and the like.

As mentioned above, the present invention can include one, two, three, four, or more GalNAc molecules. If more than one GalNAc molecule is used, a single linker can be utilized that has an anchor and a plurality of branches. The anchor can be bound to the nucleic acid sequence and the plurality of branches can branch from the anchor. A respective GalNAc molecule can be bonded to each branch. Accordingly, if two GalNAc molecules are utilized, the linker can include one anchor and two branches, whereas if three GalNAc molecules are utilized, the linker can include one anchor and three branches, and so on.

In certain embodiments, the present invention can utilize more than one linker for more than one GalNAc molecule. For example, for each GalNAc molecule, the conjugated composition can include a corresponding linker. Each linker can bind to a different nucleotide of the nucleic acid sequence and a respective GalNAc molecule can bind to an end of each respective linker.

An example of a nucleic acid conjugated to GalNAc molecules by a linker or more than one linker is described in Rajeev et al. “Hepatocyte-Specific Delivery of siRNAs Conjugated to Novel Non-nucleosidic Trivalent N-Acetylgalactosamine Elicits Robust Gene Silencing in Vivo”, ChemBioChem 2015, Volume 16, Issue 6, p. 903-908, which is incorporated herein by reference in its entirety.

In certain embodiments, the nucleic acid sequence is indirectly conjugated to the at least one GalNAc molecule by a cationic or neutrally charged polymer, and, for example, can be in the form of an encapsulated nucleic acid particle. The nucleic acid sequence can be a nanoparticle encapsulated by the cationic or neutrally charged polymer that is conjugated with GalNAc and/or its derivatives. The cationic charged polymer of the present invention can be poly(ornithine), poly(brene), poly(arginine), poly(L-lysine), poly(amidoamine), poly(ethylene imine), or the cationic charged polymer described in U.S. Pat. No. 9,943,608 B2 and Zhao “Development of a Novel Gene Delivery Toolkit Using Star-Shaped Cationic Polymers” The Graduate School of Biomedical Sciences, Baylor College of Medicine, Houston, Texas, Apr. 11, 2018, both of which are incorporated herein by reference in its entirety.

A cationic or neutrally charged polymer can refer to a chemical structure that includes a plurality of polymeric arms. A linker can be associated with each of the plurality of polymeric arms. For example, the linker is covalently associated with a plurality of polymeric arms. The linker can be non-covalently associated with a plurality of polymeric arms. For example, non-covalent association can be through one or more of ionic interactions, acid-base interactions, hydrogen bonding interactions, pi-stacking interactions, van der Waals interactions, adsorption, physisorption, self-assembly, sequestration, hydrophobic interactions, and combinations thereof.

In certain embodiments, the linker is a chemical structure with multiple functional groups (e.g., amine groups) that can covalently couple to polymers. The linker can include small molecules, macromolecules, nanoparticles, and combinations thereof. The linker can include a nanoparticle, such as a nanoparticle functionalized with a plurality of functional groups. The functional groups can include at least one of amine groups, hydroxyl groups, carboxylic acid groups, azide groups, thiol groups, carbonyl groups, alkyne groups, alkene groups, halogens, activated esters, and combinations thereof.

The cationic or neutrally charged polymer can have various types of shapes when associated with polymeric arms. For example, the polymer units have dendritic shapes, star-like shapes, brush-like shapes, comb-like shapes, circular shapes, or combinations of one or more of the above shapes. For example, the cationic or neutrally charged polymer can have hybrid structures that include a star-like shape and a brush-like shape. In certain embodiments, the cationic or neutrally charged polymer is considered a star polymer (SP) having the star-like shape.

The cationic or neutrally charged polymer can also have various molecular weights. For example, the cationic or neutrally charged polymer can have a molecular weight (Mₙ) ranging from 1 kDa to 500 kDa.

Polymeric arms of the present invention generally refer to individual polymers that are associated with the cationic or neutrally charged polymer. The polymeric arms can include biodegradable polypeptides, polysaccharides, or any combination of said polymer blocks. For example, polypeptides can be derived from cationic amino acids, cationic amino acid derivatives, and combinations thereof. The cationic amino acids and cationic amino acid derivatives can include at least one of lysine, ornithine, histidine, arginine, serine, threonine, cysteine, aspartic acid, glutamic acid, tyrosine, 2,4-diaminobutyric acid, and combinations thereof. The polymeric arms can include a biodegradable polypeptide backbone with pendant cationic side chains.

The polymeric arms can include asparagine based polymers. The polymeric arms can include derivatives of aspartic acid (i.e., poly(aspartic acid) derivatives). In some embodiments, the poly(aspartic acid) derivatives in the polymeric arms include multiple units of aspartic acid derivatives. The polymeric arms can include, for example, from 2 units to 500 units of aspartic acid derivatives.

The cationic or neutrally charged polymer can be associated with a number of polymeric arms. For example, the cationic or neutrally charged polymer can include from 3 polymeric arms to 200 polymeric arms, from 3 polymeric arms to 50 polymeric arms, from 4 polymeric arms to 20 polymeric arms, from 5 polymeric arms to 15 polymeric arms, from 6 polymeric arms to 10 polymeric arms, or at least 8 polymeric arms.

The nucleic acid sequence of the conjugated composition can be associated with a cationic or neutrally charged polymer, and the at least one GalNAc molecule, to form a polyplex that can be considered an encapsulated nucleic acid particle. For example, a star polymer bonds to the at least one GalNAc molecule and encapsulates the nucleic acid particle by electrostatic forces. The at least one GalNAc molecule can bond to the star polymer by a covalent bond.

The star polymer can encapsulate the nucleic acid particle by at least partially or fully surrounding the nucleic acid particle. The star polymer can encapsulate the at least one GalNAc molecule by at least partially or fully surrounding the at least one GalNAc molecule. As an example, the at least one GalNAc molecule and/or the nucleic acid particle can be considered disposed at an interior of the polyplex, i.e., the arms of the star polymer are folded inwardly due to electrostatic forces towards one another with the at least one GalNAc molecule and/or the nucleic acid disposed on an inner surface of the arms of star polymer. The conjugated composition, including the encapsulated nucleic acid particle, can then interact with a cell, such as a liver cell.

In certain embodiments, a nucleic acid sequence can be bonded to each of the polymeric arms. For example, on one side of each arm, amines such as ethylene diamine, or oligomers of amines, are bonded, which act as anchors on the surface of a supercoiled DNA minicircle through N—P bonding. The nucleic acid sequences can also be associated with polymeric arms by electrostatic interactions. In certain embodiments, the nucleic acid sequences are associated with the polymeric arms through one or more of ionic interactions, acid-base interactions, hydrogen bonding interactions, pi-stacking interactions, van der Waals interactions, adsorption, physisorption, self-assembly, sequestration, hydrophobic interaction, and combinations thereof.

In certain embodiments, at least one GalNAc molecule can be bonded to each of the polymeric arms. For example, at least one GalNAc molecule is bonded by a respective linker to the other side of each arm (opposite of the side that is bonded to the nucleic acid sequence). The linkers can comprise hydrocarbon chains, ethers, esters, or other linear molecules. The at least one GalNAc molecule bonded to each polymeric arm can include at least two GalNAc molecules, at least three GalNAc molecules, or at least four GalNAc molecules.

In certain embodiments, the at least one GalNAc molecule and the nucleic acid particle are bonded to the same side of the polymeric arms.

A chemical formula for a retrosynthesis of the combination of GalNAc molecules and the star polymer can include the following:

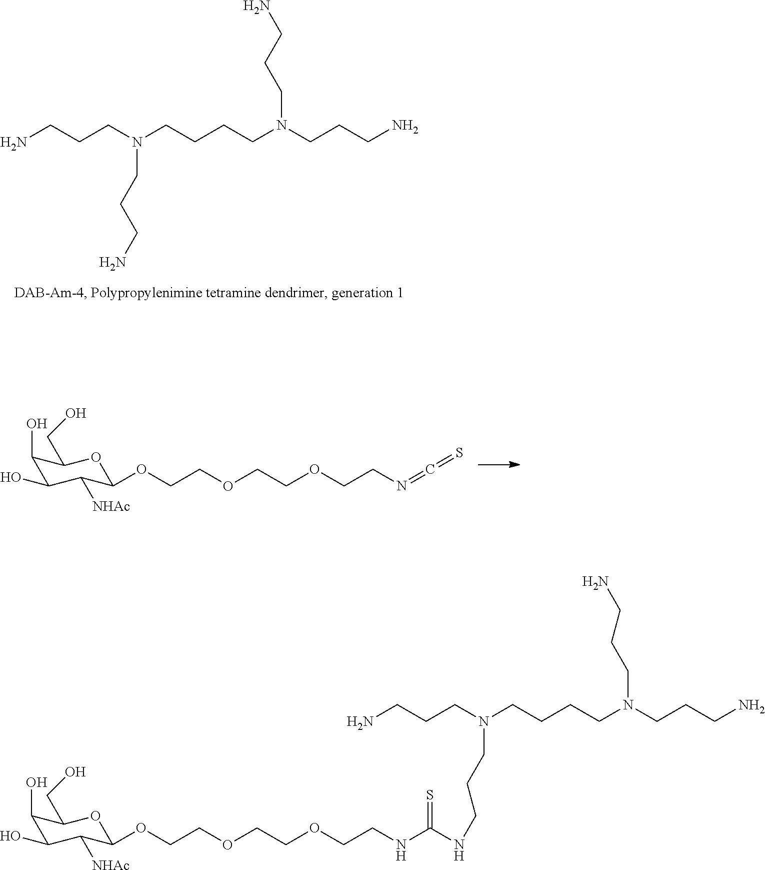 [SMILES: NCCCN(CCCN)CCCCN(CCCN)CCCN ; CC(=O)NC1C(OCCOCCOCCN=C=S)OC(CO)C(O)C1O ; CC(=O)NC1C(OCCOCCOCCNC(=S)NCCCN(CCCN)CCCCN(CCCN)CCCN)OC(CO)C(O)C1O]

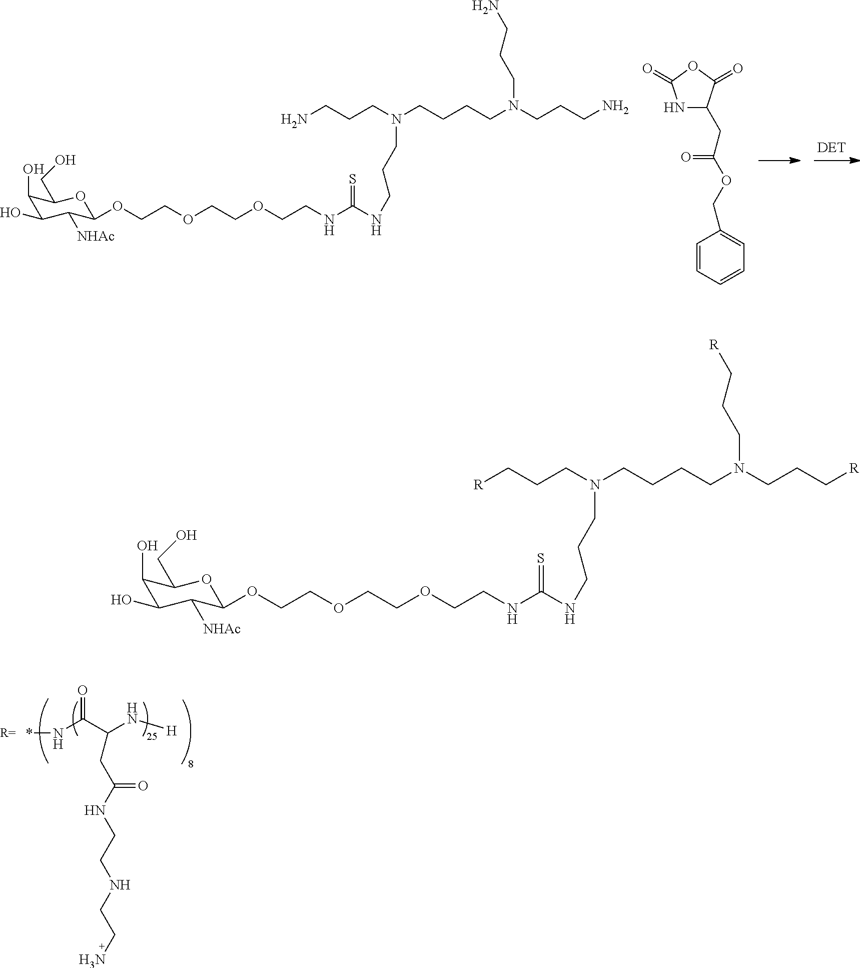

wherein two or less of the Rs can be independently selected from hydrogen, an alkene group, alkaline group, an alkyl group, a hydroxyl group, a straight or branched hydrocarbon group having from 1-20 carbons, or the like.

Another chemical formula for a retrosynthesis of the combination of GalNAc molecules and the star polymer can include the following, wherein the core is the amine skeleton shown in the Examples below:

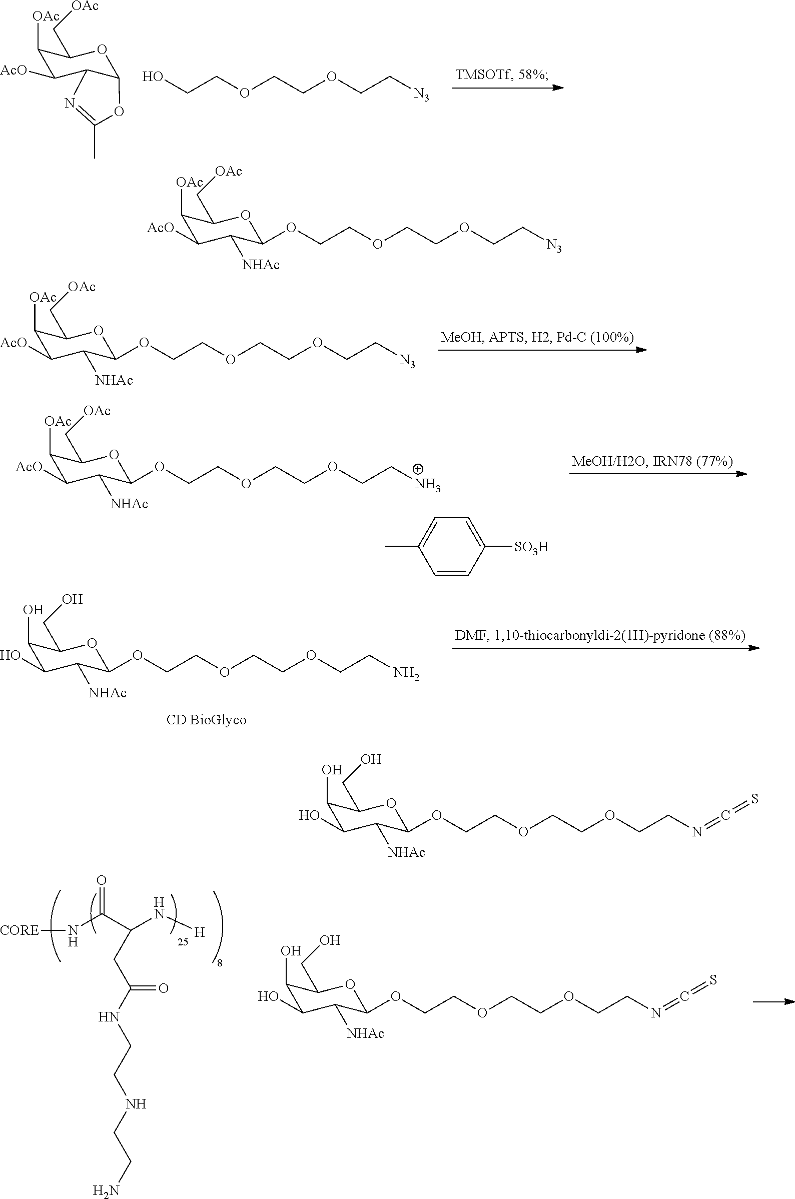

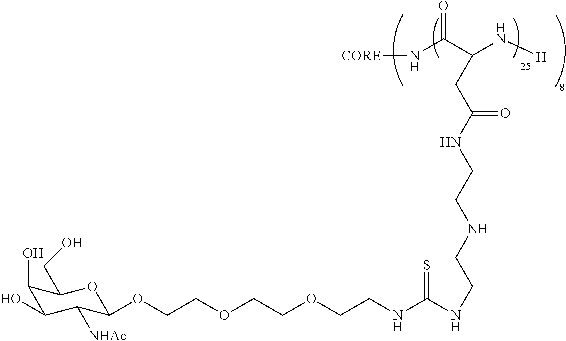

Another chemical formula for a retrosynthesis of the combination of GalNAc molecules and the star polymer can include the following, wherein the core is the amine skeleton:

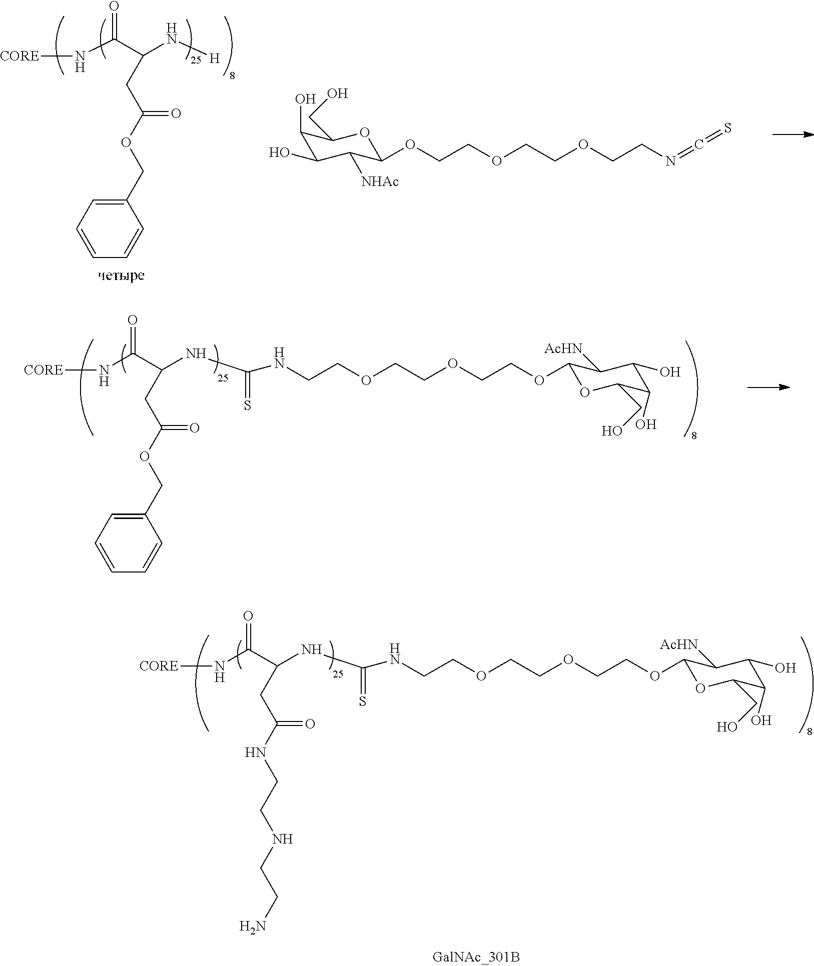

The encapsulated nucleic acid particle can have a length of from 10 nm to 500 nm, from 20 nm to 400 nm, from 30 nm to 300 nm, from 40 nm to 250 nm, from 50 nm to 220 nm, from 60 nm to 200 nm, from 80 nm to 180 nm, from 100 nm to 150 nm, or from 120 nm to 130 nm, such as from 20 nm to 300 nm. The length of the encapsulated nucleic acid particle is the longest dimension of the encapsulated nucleic acid particle. The length of the encapsulated nucleic acid particle can be less, equal to, or greater than the nucleic acid independent of encapsulation due to potential distortion of the nucleic acid when encapsulated. The small size of encapsulated nucleic acid particles ensure that the particles have a fast transport through the delivery media such as organs, tissues, and cells.

The encapsulated nucleic acid particle can have a polydispersity index value of 0.1 to 0.3, 0.15 to 0.25, or 0.2. The polydispersity index can be calculated using the following equation: PDI=(St Dev/Z Mean)\hat{ }2

The cationic or neutrally charged polymer facilitates endosomal escape via a variety of mechanisms. For example, the cationic or neutrally charged polymer facilitates endosomal escape by way of a proton sponge. The proton sponge is due to the protonation of the amino groups within the acidic endosomal lumen, in which a massive vesicular ATPase-driven proton accumulation followed by passive chloride influx into endosomes causes osmotic swelling and subsequent endosome disruption. An example of the proton sponge effect is shown in Bus et al., “The great escape: how cationic polyplexes overcome the endosome barrier”, Journal of Materials Chemistry B, 2018, pp. 6904-6918, which is incorporated herein by reference in its entirety.

Once inside the cell, the therapeutic nucleic acid can then be imported into the nucleus of the cell to treat that cell. The cationic or neutrally charged polymer fuses with the nuclear envelope and ultimately releases the encapsulated nucleic acid particle into the nucleus. Cationic or neutrally charged polymers are shown to deliver DNA to the nucleus in Godbey et al., “Tracking the intracellular path of poly(ethylenimine)/DNA complexes for gene delivery” Proc. Natl. Acad. Sci., 1999, pp. 5177-5181, which is incorporated herein by reference in its entirety.

In certain embodiments, a variety of groups that actively or passively target cells can be bonded to the surface of the cationic or neutrally charged polymer or the supercoiled DNA minicircle. For example, such groups include, but are not limited to, sugars, high-density cholesterols, and other carbohydrates. The above-mentioned groups can aid in the attachment of the conjugated composition of the present invention to high metabolism cells, such as cancerous cells, fibrotic cells, and the like.

In certain embodiments, the nucleic acid sequence is directly conjugated to the at least one GalNAc molecule by a linker, and all three components are considered parts of the conjugated composition. One example of conjugating a nucleic acid sequence directly to the at least one GalNAc molecule is by using a linker that includes a triplex. A first nucleic acid sequence can be bonded to a particle surface of a second nucleic acid sequence, such as the particle surface of a supercoiled DNA minicircle, to link the first and second nucleic acids together.

Triplexing is the formation of a triple-stranded DNA (also known as H-DNA or Triplex-DNA). The triple-stranded DNA is a DNA structure in which three oligonucleotides wind around each other and form a triple helix. In triple-stranded DNA, the third strand binds to a B-form DNA double helix (via Watson-Crick base-pairing) by forming Hoogsteen base pairs or reversed hydrogen bonds.

DNA is normally found and isolated as a double-stranded helical molecule. However, when the strand is negatively supercoiled or underwound, the strand can adopt different configurations. One such configuration is the triple-stranded DNA. In this situation, a third oligonucleotide binds to a normal double-stranded helix in a way where there is hydrogen bonding occurring among the bases of all three strands.

To form a triple-stranded DNA, an oligonucleotide can be modified internally and conjugated with GalNAc by a linker. The modified oligonucleotide is the third helix. Triplex forming oligonucleotides (TFOs) bind to the major groove of a duplex DNA. There are sequence preferences for TFOs, notably homopurine-homopyrimidine regions.

As described in detail above, the supercoiled DNA minicircle is a double stranded helix and thus is the B-form DNA double helix of the triple-stranded DNA. The double stranded helix can include certain nucleic acid sequences that are exposed at the surface of the particle. The at least one GalNAc molecule is bonded to an oligonucleotide, such as a DNA oligonucleotide, by a linker. The linker can be any of the linkers described herein. The oligonucleotide has a sequence counterpart to the exposed nucleic acid sequence of the supercoiled DNA minicircle, that is, the oligonucleotide has a sequence that bonds to the exposed nucleic acid sequence of the supercoiled DNA minicircle. The oligonucleotide, that is also bonded to the at least one GalNAc molecule, forms the third strand of the triple-stranded DNA.

The bonding of the oligonucleotide with the exposed nucleic acid sequence of the supercoiled DNA minicircle results in an embodiment of the conjugated composition of the present invention, which has a local triplex (triple-stranded DNA), as illustrated in FIG. 1. The local triplex is defined as the portion of the conjugated composition that is triple-stranded DNA, while the remaining portion of the supercoiled DNA minicircle is double-stranded DNA. The bonding between the oligonucleotide and the supercoiled DNA minicircle effectively grafts the GalNAc to the supercoiled DNA minicircle, resulting in the conjugated composition.

The oligonucleotide has a sequence that is designed to match with a selected exposed portion of the supercoiled DNA minicircle, which portion does not affect the transcription of the supercoiled DNA minicircle when the supercoiled DNA minicircle is within the living cell. For example, the oligonucleotide can be placed on the supercoiled DNA minicircle away from important sequences such as the promoter and therapeutic sequences. The location can be spaced apart from such important sequences by a distance of from 5 to 1000 bases or base pairs, or a distance of from 20 to 500 bases or base pairs, or a distance of from 50 to 300 bases or base pairs, or a distance of from 100 to 200 bases or base pairs, or 150 bases or base pairs.

The presence of GalNAc molecules facilitates the delivery of the supercoiled DNA minicircle to the liver cell. The bond between the oligonucleotide and the supercoiled DNA minicircle is reversible through hydrogen bonding. Any possible negative consequences, such as cellular processing, cytotoxicity, immunogenicity, or the like, resulting from the oligonucleotide, is minimized because of the location of the oligonucleotide bond relative to the promoter and therapeutic sequences.

The length of the oligonucleotide can be less than 50 bases, less than 40 bases, less than 30 bases, less than 20 bases. For example, the length of the oligonucleotide can be from 15 bases to 100 bases, from 20 to 80 bases, from 20 to 60 bases, from 30 to 50 bases, or 40 bases.

In certain embodiments, the at least one GalNAc molecule that is bonded to the oligonucleotide comprises at least two GalNAc molecules. In certain embodiments, the at least one GalNAc molecule that is bonded to the oligonucleotide comprises at least three GalNAc molecules or more.

The conjugated composition with the local triplex can have a length of from 10 nm to 500 nm, from 20 nm to 400 nm, from 30 nm to 300 nm, from 40 nm to 250 nm, from 50 nm to 220 nm, from 60 to 200 nm, from 80 nm to 180 nm, from 100 nm to 150 nm, or from 120 nm to 130 nm. The small size of conjugated composition with the local triplex ensures that the particles have a fast transport through the delivery media such as organs, tissues, and cells.

FIG. 1 provides a schematic diagram of three GalNAc molecules bonded to a linker. The linker comprises moieties Y and Z, three moieties X, a phosphate group, and a DNA oligonucleotide 12. In FIG. 1, each of X and Z, is independently ethylene glycol, an alkyl spacer, or the like, and Y is a multifunctional moiety allowing branching of the cluster of three GalNAc molecules.

The phosphate group provides an oxygen bridge to Z and an oxygen bridge to DNA oligonucleotide 12. DNA oligonucleotide 12 is grafted to the surface of the supercoiled DNA minicircle 10 through triplexing.

In certain embodiments, the at least one GalNAc molecule is directly conjugated to the nucleic acid sequence without forming a triple-stranded DNA. The at least one GalNAc molecule can be directly conjugated to any chemical group of the nucleic acid sequence, for example, a phosphate group, a deoxyribose sugar, a nitrogenous base, or the like, by using a linker as described herein. The at least one GalNAc molecule can be directly conjugated to the nucleic acid sequence by using nicking enzymes.

FIG. 2 illustrates another exemplary structure for bonding the supercoiled DNA minicircle 10 to the at least one GalNAc molecule. The double helix of the supercoiled DNA minicircle 10 has an exposed phosphate group on its surface. The at least one GalNAc molecule is bonded to a linker and the linker is directly bonded to the exposed phosphate group on the surface of the supercoiled DNA minicircle 10. As shown in FIG. 2, three GalNAc molecules are bonded to the supercoiled DNA minicircle 10 by a linker. The linker comprises an oxygen bridge, moieties Z and Y, and three moieties X, with each moiety X being bonded to a respective GalNAc molecule. In regard to the linker, each X, and Z, is independently ethylene glycol, an alkyl spacer, or the like, and Y is a multifunctional moiety allowing branching of the cluster of three GalNAc molecules.

In certain embodiments, the supercoiled DNA minicircle can be negatively-supercoiled or underwound, such that the strands are easily pulled apart from one another, allowing the bases of the double helix to flip outwardly. The at least one GalNAc molecule can directly bond with one of the flipped-out bases, through the linker. Due to the presence of the GalNAc molecule, the conjugated composition first binds to a liver cell receptor and then is taken up by one or more endosomes of the cell, wherein the conjugated composition dissociates from the receptor.

Once the conjugated composition is inside the cell, the linker is broken or cleaved through chemical or enzymatic means. Following this cleavage, if the DNA base is returned to its native state, then it will flip back inside the helix and normal DNA metabolism can proceed. If the DNA base is altered due to being underwound, then altered DNA is recognized by the cell as damaged and is repaired by the base excision repair (BER) pathway. During the BER pathway, the damaged base is first removed by a glycosylase, forming a DNA lesion. An exonuclease then acts to remove a few more bases around the DNA lesion. Then, DNA polymerase fills in the gap and DNA ligase seals the final nick. The DNA is thereby returned to its native state and normal DNA metabolism in the liver cell proceeds.

In certain embodiments, the nucleic acid sequence is nicked and the at least one GalNAc molecule is bonded to the nucleic acid sequence at the nicked region. Methods of nicking nucleic acid sequences are described, for example, in Luzzietti et al., “Efficient preparation of internally modified single-molecule constructs using nicking enzymes”, Nucleic Acid Research, 2011, Vol. 39, No. 3, and described in Luzzietti et al., “Nicking enzyme-based internal labeling of DNA at multiple loci”, Nature Protocols, 2012, Vol. 7, No. 4, pp 643-653, both of which are incorporated herein in their entireties by reference.

In an example, the supercoiled DNA minicircle can be nicked with a sequence-specific endonucleic nickase. The free 5′ or 3′ end of the nick is available for direct conjugation. With the correct moiety, at least one GalNAc molecule with a cleavage linker can be attached to either the 5′ or 3′ end and then is still able to relegate and seal the nick. By rescaling the nick, the supercoiling of the DNA minicircle can be re-established. Once the conjugated supercoiled DNA minicircle is taken up by the liver cell, the linker can be cleaved.

The nick is placed on the supercoiled DNA minicircle away from important sequences such as the promoter and therapeutic DNA sequences. Even if there is a remnant of the linker attached to the backbone of the supercoiled DNA minicircle, because the linker is located away from the important sequences, the impact of the remnant is minimal.

Another nicking method that can be used according to the present invention involves producing a plurality of nicking sites. The plurality of nicking sites can be produced in tandem on the supercoiled DNA minicircle, and can include, for example, from 2 to 6 nicking sites, from 3 to 5 nicking sites, or 4 nicking sites. The locations of the nicking sites can be spaced apart from the important sequences, such as the promoter sequence and any therapeutic gene sequences of the supercoiled DNA minicircle. The location of the nicking sites can be spaced apart from such important sequences by a distance of from 5 to 1000 bases or base pairs, or a distance of from 20 to 500 bases or base pairs, or a distance of from 50 to 300 bases or base pairs, or a distance of from 100 to 200 bases or base pairs, or 150 bases or base pairs.

The nickase (nicking enzyme) used in the nicking method can be Nt.BbvCI, Nb.BbvCI, or the like. The nickase can be used to generate nicks every 2 to 12 bases, every 4 to 10 bases, or every 6 to 8 bases, depending on the type of nickase used.

To create the conjugated composition of the present invention using the nicking method, a single-stranded oligonucleotide is modified internally (i.e., away from the ends of the strand) and conjugated with GalNAc. Being internally conjugated, the ends of the oligonucleotide are unchanged. A sample of the nickase, the supercoiled DNA minicircle, and the single-stranded oligonucleotide DNA are combined and slightly heated to 80° C. for 2 minutes. The elevated temperature causes multiple nicked pieces in tandem to unhybridize, leaving a single-stranded gap. The single-stranded oligonucleotide is exactly the same size as the single-stranded gap and thus is longer than the multiple nicked pieces. The sample is then cooled to 20° C. at a rate of 1° C./min to allow the longer single-stranded oligonucleotide conjugated with GalNAc to anneal into the created gap in the supercoiled DNA minicircle.

The conjugated composition now includes at least one GalNAc molecule and the therapeutic gene. Once the conjugated supercoiled DNA minicircle is taken up by the liver cell, the linker can be cleaved. The nicks are placed on the supercoiled DNA minicircle away from important sequences, for example, away from a promoter sequence and away from any therapeutic gene sequences. Even if there is a remnant of the linker attached to the backbone of the supercoiled DNA minicircle, the impact of the remnant would be minimal because the linker is located away from the important sequences.

The conjugated composition comprising at least one GalNAc conjugated to the supercoiled DNA minicircle, can include additional conjugates, by way of nicking. For example, the conjugated composition can also be treated with intercalators, such as ethidium bromide, chloroquine, or the like, to modulate the conjugated supercoiled DNA minicircle's degree of negative supercoiling. Proteins, such as the archaeal histone-like protein HmfB from Methanothermus fervidus can be used to generate positive supercoiling. The amount of negative or positive supercoiling can modulate the overall shape of the DNA molecule, which may be used to control transfection efficiency and specific cell type uptake. Once inside the cell, the native topoisomerases will relax the DNA minicircle to the normal steady state level of negative supercoiling.

The present invention also provides a method of treating a patient, such as a human or other animal, by administering a conjugated composition as described herein. The conjugated composition can be any of the conjugated compositions described herein, or comprise different combinations of the features of the conjugated compositions described herein. The conjugated composition used in the method at least includes a therapeutic nucleic acid sequence directly or indirectly conjugated to at least one GalNAc molecule, or to a derivative of a GalNAc molecule, by a linker. The conjugated composition can be administered to a patient by subcutaneous administration, intravenous administration, intramuscular administration, intraperitoneal administration, oral administration, topical administration, transdermal administration, vaginal administration, suppository, by inhalation, or the like.

The present invention also provides a method of contacting a living cell, for example, a hepatocyte, with a conjugated composition as described herein. The conjugated composition at least includes a therapeutic nucleic acid sequence that is directly or indirectly conjugated to at least one GalNAc molecule, or to a derivative of a GalNAc molecule, by a linker. The at least one GalNAc molecule of the conjugated composition then binds to an ASGPR of the living cell. The conjugated composition is then taken up in an endosome of the cell, wherein the conjugated composition dissociates from the ASGPR. The at least one GalNAc molecule is then cleaved from the therapeutic nucleic acid sequence, and the therapeutic nucleic acid sequence enters the nucleus where it exerts its therapeutical effects to treat a living cell.

The present invention further includes, in combination, a treated cell and a remnant GalNAc molecule including remnants of a linker. The treated cell includes a human liver cell and a synthetic nucleic acid sequence metabolized in the human liver cell, the synthetic nucleic acid sequence having been formed from cleavage of a linker of a conjugated composition as described herein. The conjugated composition can include a synthetic nucleic acid sequence directly or indirectly conjugated to a GalNAc molecule, by the linker. The remnant GalNAc molecule includes the GalNAc molecule of the conjugated composition, and a remnant of the cleavable linker attached thereto.

The present invention improves the ability to deliver polymer-encapsulated nucleic acids, such as to liver cells, while retaining polyplex particle sizes less than 150 nm and as small as 40 nm or less, thereby achieving more uniform treatment of the liver.

The present invention also improves the ability to deliver small naked circular DNA nanoparticles to liver cells without the need for an encapsulating agent (e.g., lipid, silicon, silica, or polymer nanoparticle).

The incidence of GalNac-induced endocytosis in liver cells grows non-linearly with the number of conjugated GalNac molecules. The plurality of arms afforded by a branched polymer exhibiting a plurality of arms affords increased opportunity to grow the number of GalNac groups conjugated to it thereby improving cellular uptake.

The present invention will be further clarified by the following examples, which are intended to be exemplary of the present invention.

### EXAMPLES

### Example 1: Preparation of 8-Arm Tosylated Tripentaerythritol

A 6 M HCl (CAS No.: 7647-01-0 Fisher Chemical) solution and purified water was prepared and stored at 0° C. before experiments for further use. A 250 mL round-bottom flask with a magnetic bar was sealed by a septum stopper and connected to a Schlenk Line through a metal needle. The round-bottom flask was flamed and degassed by the Schlenk line for three times. 3.7 g of tripentaerythritol (CAS No.: 78-24-0 Sigma-Aldrich) was added into the flask and degassed three times. 50 mL of anhydrous pyridine (CAS No.: 110-86-1 ReagntPlus) was injected into the flask to suspend the tripentacrythritol. Then, 19 g of p-toluenesulfonyl chloride (CAS No.: 98-59-9 Sigma-Aldrich) was dissolved in an extra 30 mL of anhydrous pyridine and injected into the suspension, the color changed from light brown to bright yellowish color.

The mixture was kept at ambient temperature under stirring and argon bubbling for 24 h. The mixture solution became milky brown color. The mixture was poured into cooled HCl (300 mL, 6M) (CAS No.: 7647-01-0 Fisher Chemical) to precipitate the white powder and the powder was filtered and washed with water and methanol (Sigma-Aldrich) to achieve the crude products. There was a considerable amount of product remaining in the aqueous mother solution, and further extraction was performed. In addition, a considerable amount of insoluble solid stayed in the aqueous layer, which was extracted with CHCl₃ (Sigma-Aldrich).

The following steps were repeated multiple times. The achieved crude precipitate was dissolved in CHCl₃ and gently extracted with water and brine, the filtrate organic layer was collected in a 500 mL bottle and dried on Na₂SO₄ and sorted in 0° C. for 2 hours. Na₂SO₄ in the mixture was discarded by filtration. Extra organic solvent in the filtrate was evaporated under reduced pressure to obtain a viscous colorless oil. Dried product was collected into a pre-flamed and degassed round-bottom flask. 100 mL of anhydrous MeOH was injected into the flask to dissolve the product at 40° C. Then, 10 mL of anhydrous CHCl₃ was injected into the flask and temperature was increased to 55° C. until a clear solution was achieved. The clear solution was stayed in 0° C. temperature overnight to obtain white powder precipitation. The remaining solvent was removed and the white crystal was further recrystallized twice using MeOH/CHCl₃ by repeating the step of collecting dried product into the pre-flamed and degassed round-bottom flask, injecting 100 mL of anhydrous MeOH into the flask to dissolve the product at 40° C., injecting 10 mL of anhydrous CHCl₃ into the flask, and increasing the temperature to 55° C. until a clear solution was achieved. The filtrate was dried under vacuum to get purified 8-arm tosylated tripentaerythritol (25% yield).

The following reference for the preparation of 8-Arm Tosylated Tripentaerythritol was used. To tripentaerythritol (3.7 g, 0.01 mol) suspended in pyridine (50 mL), p-toluenesulfonyl chloride (19 g, 0.10 mol) was added at 0-5° C. After 1 h, the reaction mixture was warmed up to room temperature, and stirred for another 24 h. The mixture was poured slowly into a beaker containing HCl (6 M, 300 mL). A large amount of white precipitate was collected by filtration, washed with water and methanol. The precipitate was sonicated in chloroform (200 mL), transferred to a separatory funnel and water (200 mL) was added. A considerable amount of insolubles stayed in the aqueous layer, which was extracted twice with chloroform. The organic phase was combined, washed with water and brine, dried and evaporated that resulted in a viscous colorless oil (˜5 mL). After recrystallizing twice in methanol and completely drying, a foam-like colorless solid was obtained (3.0 g, 19% reported, yield 25%).

¹H Nuclear magnetic resonance spectroscopy (NMR) (400 MHZ, CDCl₃) results included the following: 7.72 (t, 16H, J=8.8 Hz, 8.0 Hz), 7.37 (t, 16H, J=6.8 Hz, 8.0 Hz), 3.81 (s, 16H), 3.19 (s, 4H), 3.14 (s, 4H), 2.44 (s, 24H). 13C NMR (100 MHZ, CDCl₃) results included the following: 145.4, 145.3, 145.2, 132.3, 131.9, 130.1, 128.0, 68.472, 67.7, 67.5, 66.6, 44.3, 43.8, 21.7. Mass Spectrometry (MS) (MALDI-TOF, using dithranol and sodium acetate as the matrix) results included the following: m/z [M+Na]⁺ 1627.02.

The following chemical equation represents the reaction taking place in Example 1:

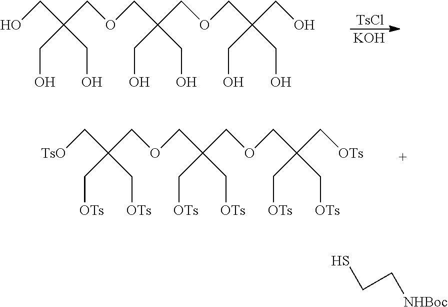

### Example 2: Preparation of 8-Arm Boc-Protected Amine

A 0.2 M HCl solution and purified water was prepared and stored at 0° C. before experiments for further use. A 50 mL round-bottom flask with a magnetic bar was sealed by septum stopper and connected to a Schlenk Line through a metal needle. The round-bottom flask was flamed and degassed by the Schlenk line three times. 2 g of 1-butyl 2-mercaptoethylcarbamate (CAS No.: 673-09-5 Sigma-Aldrich) was added into the flask and degassed three times. 5 mL of anhydrous N, N-Dimethylformamide (DMF) (CAS No.: 68-12-2 Sigma-Aldrich) was injected into the flask to suspend 1-butyl 2-mercaptoethylcarbamate at 0° C. Then, while remaining at 0° C., 375 mg NaH (CAS No.: 7646-69-7 Sigma-Aldrich) was added into the suspension. The mixture was kept at 0° C. under stirring and argon for 10 min. 5 mL of anhydrous DMF was injected into the flask to suspend 1.62 g of 8-arm tosylated tripentarythritol (Example 1) that was degassed three times. The solution of 8-arm tosylated tripentarythritol in dry DMF (5 mL) was added slowly into the mixture for 15 min., and the mixture was allowed to stir overnight under nitrogen at room temperature.

The mixture was poured into a cooled HCl (60 mL, 0.2M) solution and the aqueous solution was extracted with ethyl acetate (3×50 mL). The organic phase was rinsed with water (2×50 mL) and brine (50 mL), and dried over anhydrous sodium sulfate. Na₂SO₄ in the mixture was discarded by filtration. Extra organic solvent in the filtrate was evaporated under reduced pressure to obtain viscous foamy white crystals. The impure compound was further separated on a Yamazen Dual LC system using a combination of MeOH: DCM (1:3) as eluent. The evaporated product was collected into a pre-flamed and degassed round-bottom flask, 100 mL of anhydrous MeOH was injected into the flask to dissolve the product at 40° C. Then 10 mL of anhydrous in CHCl₃ was injected into the flask and the temperature was increased to 55° C. degree until a clear solution was achieved.

The clear solution was stayed at 0° C. overnight to obtain white powder precipitation. The remaining solvent was removed and the white crystal was further recrystallized twice using MeOH/CHCl₃ by repeating the step of collecting the evaporated product into the pre-flamed and degassed round bottom flask, injecting 100 mL of anhydrous MeOH into the flask to dissolve the product at 40° C., injecting the anhydrous CHCl₃ into the flask, and increasing the temperature to 55° C. degree until a clear solution was achieved. The filtrate was dried under vacuum to obtain purified 8-arm boc-protected amine (71%).

The following reference for the preparation of 8-arm boc-protected amine was used. A solution of t-butyl 2-mercaptoethylcarbamate (2 g, 11.3 mmol) in dry DMF (5 mL), sodium hydride (90% in mineral, 375 mg, 14 mmol) was added under nitrogen at 0° C. After 10 min, a solution of 8-arm tosylated tripentarythritol (1.62 g, 1.0 mmol) in dry DMF (5 mL) was added and the mixture was allowed to stir overnight under nitrogen at room temperature. The reaction mixture was poured into aqueous HCl (0.2 M, 60 mL), and was extracted with ethyl acetate (3× 50 mL). The organic phase was rinsed with water (2×50 mL) and brine (50 mL), dried over anhydrous sodium sulfate, filtered and evaporated. After purification on a flash column with MeOH/DCM as the eluent, a colorless solid was obtained (0.70 g, 71%).

¹H NMR (400 MHZ, CDCl₃) results included the following: 3.32 (s, 24H), 2.68 (m, 32H), 1.70 (s, 8H), 1.44 (s, 72H). 13C NMR (100 MHz, CDCl₃) results included the following: 155.8, 79.3, 73.0, 71.5, 44.5, 44.2, 40.0, 36.5, 35.2, 34.0, 33.8, 28.9, 28.4. High-resolution mass spectrometry (HRMS) results included the following: m/z [M+Na]⁺ 1667.7589.

The following chemical equation represents the reaction taking place in Example 2:

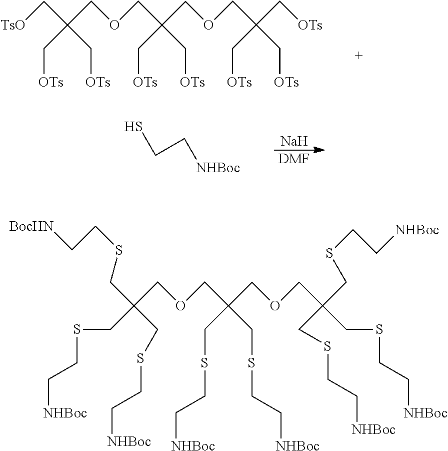

### Example 3: Preparation of 8-Arm Free Amine Initiator

A saturated sodium bicarbonate solution using purified water was prepared, filtered and stored at 0° C. before experiments for further use. A column packed with an Amberlite IRA-67 ion exchanger (10-20 gram) (CAS No. 476633-1 KG Sigma-Aldrich) was prepared. A 50 mL round-bottom flask with a magnetic bar was sealed by a septum stopper and connected to a Schlenk Line through a metal needle. The round-bottom flask was flamed and degassed by a Schlenk line three times. 550 milligrams of the 8-arm Boc-protected amine (Example 2) was added into the flask and degassed for three times. 4 mL of anhydrous chloroform (CAS Number: 67-66-3 Sigma-Aldrich) was injected into the flask to suspend the 8-arm Boc-protected amine at room temperature under nitrogen.

To the solution of 8-arm Boc-protected amine in dry chloroform, trifluoroacetic acid (2 mL) (CAS No.: 76-05-1 Reagentplus) was added slowly into the mixture for 10 min. under nitrogen, and the mixture was allowed to stir for 4 hours under nitrogen at room temperature. The mixture was evaporated under reduced pressure to obtain a viscous colorless oil. The dried colorless oil was poured into a 100 ml beaker filled with 20 mL of deionized (DI) water. The insoluble material in the solution was removed by centrifugation (13000 g for 5 minutes at 20° C.).

A column packed with Amberlite IRA-67 (CAS No.: 476633-1 KG Sigma-Aldrich) ion exchanger was treated with saturated sodium bicarbonate solution and copious amounts of water. The supernatant was neutralized by passing through the column pretreated Amberlite IRA-67 ion exchanger. The eluted solution with a pH>9.0 was collected and the combined an aqueous layer and reverse extracted with CHCl₃ (15 mL). The combined solution collected from the Amberlite IRA-67 ion exchange column was subjected to freeze-drying to obtain the 8-Arm Free Amine Initiator as a colorless foam-like solid (95%).

The following reference for the preparation of 8-arm free amine initiator was used. A mixture of the 8-arm Boc-protected amine (550 mg, 0.33 mmol) and trifluoroacetic acid (2 mL) in chloroform (4 mL) was stirred at room temperature for 4 h and evaporated to dryness. The residue was dissolved in water (20 mL) and the insoluble material was removed by centrifugation. The supernatant was neutralized by passing through a column packed with Amberlite IRA-67 ion exchanger that was pretreated with saturated sodium bicarbonate solution and copious amounts of water. The eluted solution with pH>9.0 was collected and subjected to a reverse extraction with CHCl₃ (10 mL), the obtained aqueous layer was put under freeze-drying to obtain the product as a colorless foam-like solid (250 mg, 89%).

¹H NMR (400 MHZ, CDCl₃) results included the following: 3.32 (s, 8H), 2.86 (m, 16H), 2.62 (m, 32H), 1.51 (s, 16H). 13C NMR (100 MHZ, CDCl₃) results included the following: 73.0, 71.9, 44.8, 44.3, 41.4, 41.4, 38.3, 38.0, 36.6, 36.2, 35.3. HRMS results included the following: m/z [M+H]⁺ 845.3940.

The following chemical equation represents the reaction taking place in Example 3:

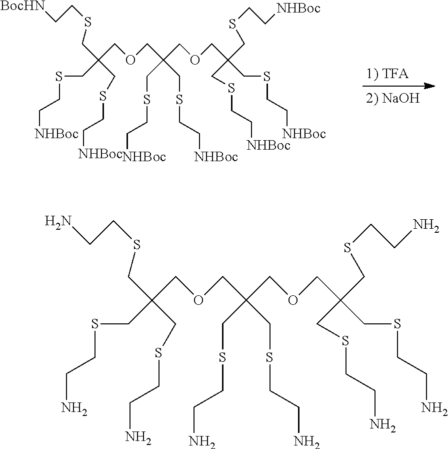 [SMILES: CC(C)(C)OC(=O)NCCSCC(COCC(CSCCNC(=O)OC(C)(C)C)(CSCCNC(=O)OC(C)(C)C)CSCCNC(=O)OC(C)(C)C)(COCC(CSCCNC(=O)OC(C)(C)C)(CSCCNC(=O)OC(C)(C)C)CSCCNC(=O)OC(C)(C)C)CSCCNC(=O)OC(C)(C)C ; NCCSCC(COCC(CSCCN)(CSCCN)CSCCN)(COCC(CSCCN)(CSCCN)CSCCN)CSCCN]

### Example 4: Preparation of N-Carboxyanhydride (NCA) of L-Aspartic Acid-β-Benzyl Ester

A 50 mL round-bottom flask with a magnetic bar was sealed by a septum stopper and connected to a Schlenk Line through a metal needle. The round-bottom flask was flamed and degassed by the Schlenk Line three times. 0.25 g of L-aspartic acid-β-benzyl ester (CAS No.: 13590-42-6 Santa Cruz Biotechnology) was added into the flask and degassed three times. 5 mL of anhydrous dimethyl sulfoxide (DMSO) (CAS No.: 67-68-5 Sigma-Aldrich) was injected, and degassed three times.

After 5 mL of anhydrous DMSO was injected into the flask to suspend L-aspartic acid-β-benzyl ester, the temperature was kept at room temperature, and 3.5 mg of the 8-arm free amine initiator (Example 3) was dissolved in an extra 1 mL of anhydrous DMSO and freeze-pump-thaw was performed three times. Then the initiator DMSO solution was injected into the suspension of the L-aspartic acid-β-benzyl ester at room temperature.

The mixture was stirred to room temperature under stirring and argon for three days. The mixture was poured into diethyl ether (40 mL) to precipitate a white sticky precipitate. The obtained precipitate was dissolved in chloroform, and then precipitated in diethyl ether again. The precipitate was once again subjected to chloroform, and ether was added until the solution became cloudy. More chloroform was added until a clear solution was obtained. The clear solution was stayed in −20° C. overnight to obtain white needle-shaped crystals. The remaining solvent was removed and the crystals were dried under vacuum to obtain the purified 8-arm product of poly(β-benzyl-L-aspartate) (92% yield).

The following reference for the preparation of poly(β-benzyl-L-aspartate) was used. Linear PBLAs were synthesized by the ring-opening polymerization of BLA-NCA initiated by N-Boc-2,2′-(ethylenedioxy) diethylamine. Briefly, solution a of N-Boc-2,2′-(ethylenedioxy) diethylamine (0.25 mL, 10 mg/mL) in dry DMSO was added to the solution of BLA-NCA (0.25 g, 1.0 mmol) in dry DMSO (5 mL) under an argon atmosphere, and stirred at room temperature for three days. The mixture was poured into diethyl ether (40 mL). The precipitate was dissolved in chloroform, precipitated in diethyl ether again and lyophilized in dichloromethane/benzene to give a white solid (78-85% yields).

The following reference for the preparation of the 8-arm poly(β-benzyl-L-aspartate) was used. The 8-arm poly(β-benzyl-L-aspartate) was synthesized in a similar way. Briefly, a solution of the 8-arm free amine initiator (1 mL, 3.5 mg/mL) in dry DMSO was added to the solution of BLA-NCA (0.25 g, 1.0 mmol) in dry DMSO (10 mL) under an argon atmosphere, and stirred at 35° C. for three days. The polymer product was obtained after multiple precipitations in diethyl ether (Yield 0.17 g, 83%).

¹H NMR (400 MHZ, DMSO-d₆) results for the linear poly(β-benzyl-L-aspartate) (PBLA) included the following: 8.18 (br), 7.25 (br), 5.0 (br), 4.6 (m), 2.8 (br), 2.8 (m), 1.3 (s). 1H NMR (400 MHZ, DMSO-d₆) results for the 8-arm PBLAs included the following: 8.18 (br), 7.25 (br), 5.0 (br), 4.6 (m), 2.8 (br).

The following chemical equation represents the reaction taking place in Example 4, wherein the core is the amine skeleton:

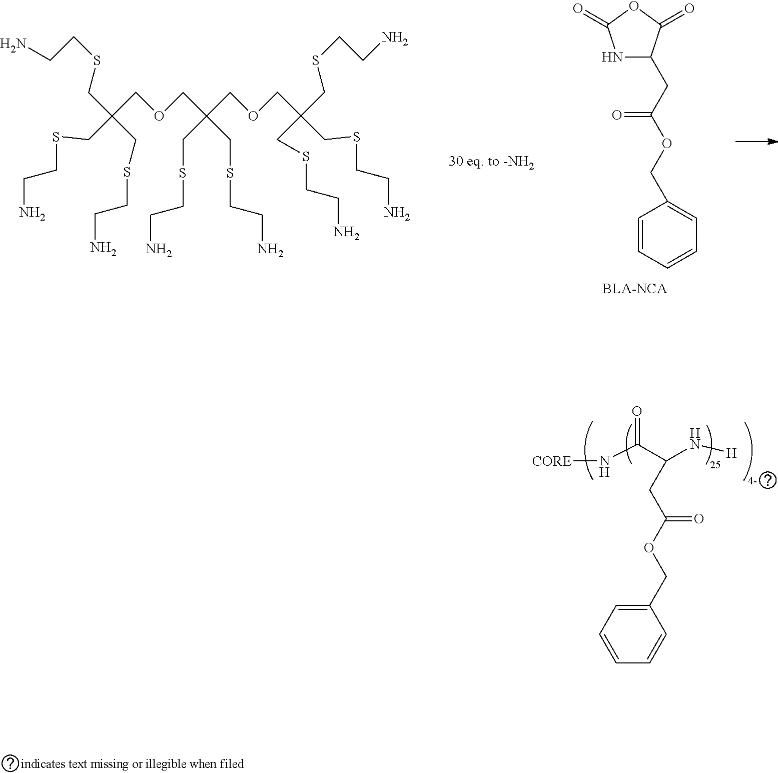

### Example 5: Preparation of N-Substituted Polyaspartamides (Star Polymer)

A 1 M and 0.01 M HCl (CAS No.: 7647-01-0 Fisher Chemical) solution and purified water were prepared and stored at 0° C. before experiments for further use. A 25 mL round-bottom flask with a magnetic bar was sealed by a septum stopper and connected to a Schlenk Line through a metal needle. The 25 mL round-bottom flask was flamed and degassed by the Schlenk line three times. The temperature was kept at 0° C. by using an ice bath.

A solution of diethylenetriamine (DET, 2.4 mL) (CAS No.: 111-40-0 Reagentplus) was prepared in ice-cold N-methyl-2-pyrrolidone (NMP) (5 mL) (CAS No.: 872-50-4 Sigma-Aldrich) before the experiment. More specifically, 2.4 mL DET was added into the 25 mL flask and degassed three times, then 5 mL of anhydrous NMP was injected under inert atmosphere at 0° C. A 50 mL round-bottom flask with a magnetic bar was sealed by a septum stopper and connected to the Schlenk Line through a metal needle. The round-bottom flask was flamed and degassed by the Schlenk line three times. The temperature was kept at 0° C. by using an ice bath.

100 mg of the star 8-arm PBLA (Example 4) was added into the flask and degassed for three times, then 5 mL of anhydrous NMP was injected under inert atmosphere. The DET, 2.4 mL/NMP (5 mL) solution was slowly added into the suspension of star 8-arm PBLA in NMP at 0° C. over a period of 10 min. The mixture was stirred at 0° C. temperature under stirring and argon for 2 hours. Then the ice-cold reaction mixture was added dropwise into 1 M HCl (50 mL) at 0° C. over a period of 30 min, and the final pH was adjusted to 4.0. The solution was dialyzed (14 kDa molecular weight cut-off membrane (CAS No.: D9777-100 FT Sigma-Aldrich) against 0.01 HCl at 4° C. for 24 h, then de-ionized water was added at 4° C. for an additional 48 h before lyophilization (90% yield).

The following reference for the preparation of the N-substituted Polyaspartamides was used. A solution of diethylenetriamine (DET, 2.4 mL) in ice-cold NMP (5 mL) was added dropwise to a solution of the star 8-arm PBLA (100 mg), and the mixture was stirred at 0° C. for additional 2 h. The ice-cold reaction mixture was added dropwise to 1 M HCl (50 mL) at 0° C. over a period of 30 min, and the final pH was adjusted to 4.0. The solution was dialyzed (14 kDa molecular weight cut-off membrane) against 0.01 M HCl at 4° C. for 24 h, and then de-ionized water at 4° C. for an additional 48 h before lyophilization. DET-modified polymers were obtained as white solids at around a 90% yield.

¹H NMR LP1 (linear polymer 1) (400 MHZ, D₂O) results included the following: 3.58 (br), 3.38 (br), 3.22 (br), 2.91 (br), 1.47 (s). 1H NMR LP2 (linear polymer 2) (400 MHZ, D₂O) results included the following: 3.58-3.38 (br), 3.21 (br), 2.95 (br), 2.79 (br). 1H NMR SP (star polymer) (400 MHZ, D₂O) results included the following: 3.42 (br), 3.17 (br), 3.02 (br), 2.88 (br). LP1 and LP2 are comparison data from Zhao (2018).

The following chemical equation represents the reaction taking place in Example 5, wherein the core is the amine skeleton:

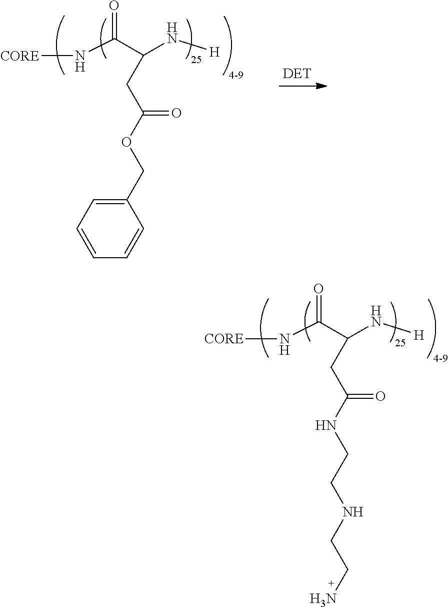

### Example 6: Preparation of β-GalNAc-PEG₃-ITC

A 25 mL round-bottom flask with a magnetic bar was sealed by a septum stopper and connected to a Schlenk Line through a metal needle. The 25 mL round-bottom flask was flamed and degassed by the Schlenk line three times. The temperature was kept at room temperature. β-GalNAc-PEG₃-amine (76 mg, 0.22 mmol) (Catalog No.: CS-3030 CarboSynUSA, Inc.) was added into the 25 mL flask and degassed for three times. 5 mL of anhydrous DMF (CAS No.: 68-12-2 Sigma-Aldrich) was injected under inert atmosphere at room temperature. 50 mg of 1,1′-Thiocarbonyldi-2 (1H)-pyridone (CAS No.: 102368-13-8 Sigma-Aldrich) was dissolved with 2 mL of DMF in the flask and degassed three times. Then 2 mL of anhydrous solution was injected into the mixture of the 25 mL round-bottom flask under inert atmosphere.

The reaction was vigorously stirred and the temperature was raised to 50° C. over a period of 10 min. The mixture was stirred at 50° C. under argon for a total of 1 hour. Then the reaction mixture was allowed to cool down to room temperature and diluted with 40 mL of DCM (Sigma-Aldrich). The organic phase was washed with water (20 mL) and brine (mL) respectively, dried over Na₂SO₄, and evaporated to dryness. Chromatography was carried out by using MeOH/DCM (1:4) (Sigma-Aldrich) as an eluent to afford 73% yield of the β-GalNAc-PEG₃-ITC.

The following preparation reference of β-GalNAc-PEG₃-ITC was used. 1,1′-Thiocarbonyldi-2 (1H)-pyridone (50 mg, 0.22 mmol) was added to a solution of β-GalNAc-PEG₃-amine (76 mg, 0.22 mmol) in dry DMF (5 mL), which was dissolved in 2 mL of DMF at ambient temperature, and the mixture was stirred at 50° C. for an additional hour. The reaction mixture was allowed to cool to room temperature, then diluted with DCM (40 ml) and washed with water (20 ml) and brine (20 ml). The organic phase was dried over Na₂SO₄ and evaporated to dryness. Preparative LC separation was carried out in a Yamazen LC using MeOH/DCM (1:4) as an eluent at around a 73% yield.

1H NMR (400 MHZ, D₂O) results included the following: 4.36 (d, 2H), 3.87 (br, 1H), 3.77 (br, 2H), 3.66 (m, 2H), 3.56 (br, 8H), 1.90 (s, 1H).

The following chemical equation represents the reaction taking place in Example 6:

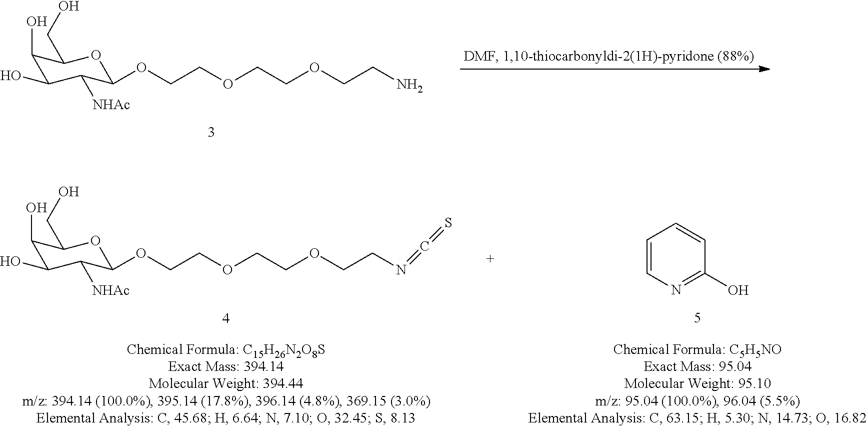 [SMILES: CC(=O)NC1C(OCCOCCOCCN)OC(CO)C(O)C1O ; CC(=O)NC1C(OCCOCCOCCN=C=S)OC(CO)C(O)C1O ; Oc1ccccn1]

### Example 7: Preparation of N-GalNAc Polyaspartamides

A tris-buffered saline (TBS) solution (CAS No.: 77-86-1) was prepared with purified water and stored at 0° C. before experiments for further use. A 25 mL round-bottom flask with a magnetic bar was sealed by a septum stopper and connected to a Schlenk Line through a metal needle. The 25 mL round-bottom flask was flamed and degassed by the Schlenk line three times. The temperature was kept at room temperature. Both the Star polymer (5, 10 mg, 0.2 nmol) of Example 5 and β-GalNAc-PEG₃-ITC (0.4 mg, 2 nmol) of Example 6 were added into the 25 mL flask and degassed for three times. 5 mL of TBS buffer solution was injected under inert atmosphere at room temperature. The reaction was vigorously stirred at room temperature under argon over a period of 1 hour duration. The mixture solution was further dialyzed (14 kDa molecular weight cut-off membrane (CAS No.: D9777-100 FT Sigma-Aldrich)) against de-ionized water at 4° C. for 4 h before lyophilization to obtain as N-GalNAc Polyaspartamides white solids (60% yield).

The following preparation reference for N-GalNAc Polyaspartamides was used. The covalent coupling of β-GalNAc-PEG₃-ITC ligands were on the capsid of the polymer via the primary amino groups. More specifically, the Star polymer (5, 10 mg, 0.2 nmol) was stirred vigorously with β-GalNAc-PEG₃-ITC (0.4 mg, 2 nmol) in TBS buffer solution (pH 9.0, 5 ml) and incubated for 1 h at room temperature. The solution was then dialyzed (14 kDa molecular weight cut-off membrane) against de-ionized water at 4° C. for 4 h before lyophilization. GalNAc-modified polymers were afforded as white solids in ˜60% yields.

¹H NMR (400 MHZ, N, N-Dimethyl-formamide-d₇) results include the following: 4.00 (q), 3.52 (br), 3.18 (br), 2.70 (br), 2.60 (br), 1.86 (s).

The following chemical equation represents the reaction taking place in Example 7, wherein the core is the amine skeleton:

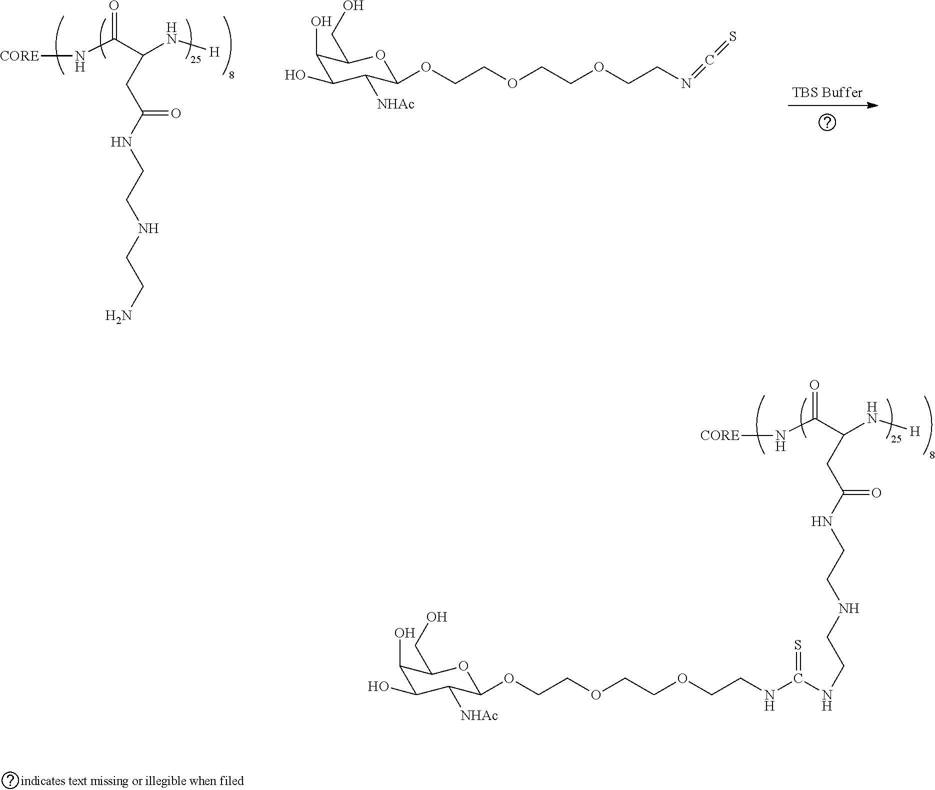

### Example 8: Preparation of Minivectors Modified with Gal-NAc (Prophetic Example)

A minivector (0.2 nmol) will be stirred vigorously with β-GalNAc-PEG₃-ITC (0.4 mg, 2 nmol) (Example 6) in TBS buffer solution (pH 9.0, 5 ml) and incubated for 12 h at room temperature. The solution will then be dialyzed against deionized water at 4° C. for 4 h before lyophilization to produce a minivector modified with Gal-NAC. The nucleophilic properties inherent in the nucleic acid backbone of the minivector will covalently attach to the isothiocyanate derivative of Gal-NAC (β-GalNAc-PEG₃-ITC).

### Example 9: Cell Viability Experiment

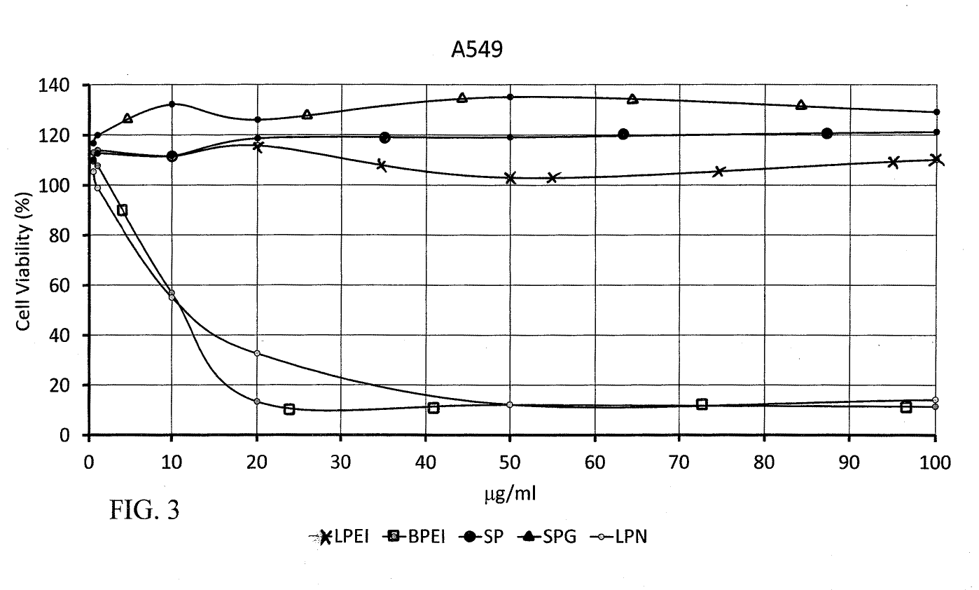

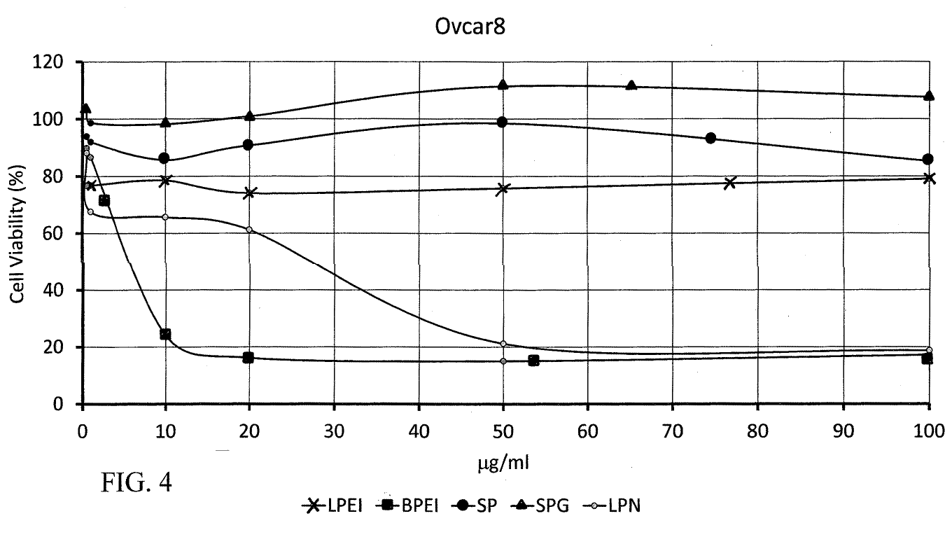

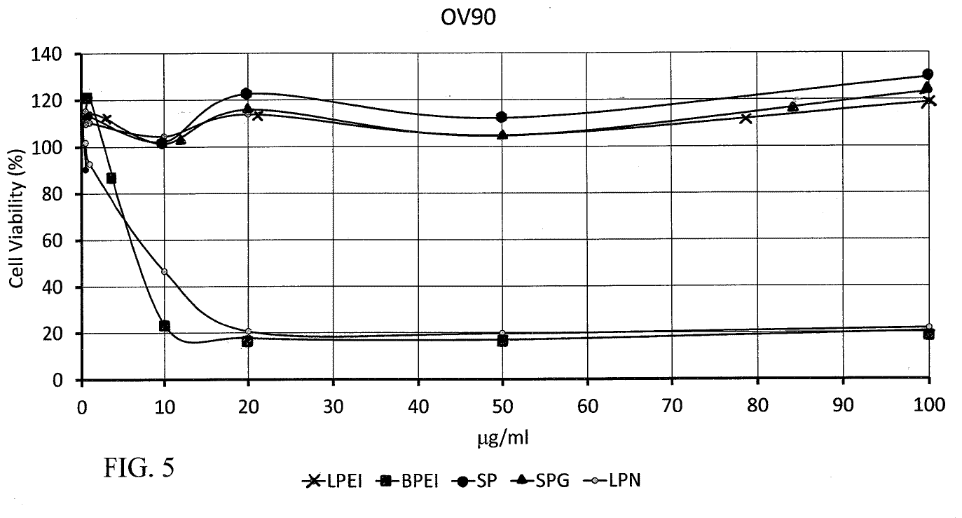

Three cell lines were tested, including A549, Ocvar8, and Ov90. Each cell line was treated with six different treatments, including linear polyethylenimine (LPEI), branched polyethylenimine (BPEI), star polymer (SP) (Example 5), star polymer Gal-NAc (SPG) (Example 7), and lipofectamine (LPN). Each of the six different treatments were applied to the cells lines at six different concentrations including 0.5 ug/ml, 1 ug/ml, 10 ug/ml, 20 ug/ml, 50, ug/ml, and 100 ug/ml. Each sample was tested three times. MTT (3-(4,5-dimethylthiazol-2-yl)-2,5-diphenyltetrazolium bromide) assays were taken 24 hours post-treatment. The MTT assay was used to measure cellular metabolic activity as an indicator of cell viability, proliferation and cytotoxicity. This colorimetric assay is based on the reduction of a yellow tetrazolium salt (MTT) to purple formazan crystals by metabolically active cells. FIGS. 3-5 provide the results of the MTT assay. As can be seen in FIG. 3, cell viability of A549 was maintained for SP and SPG at each of the six different treatment concentrations. As can be seen in FIG. 4, cell viability of Ovcar8 was maintained for SP and SPG at each of the six different treatment concentrations. As can be seen in FIG. 5, cell viability of OV90 was maintained for SP and SPG at each of the six different treatment concentrations.

### Example 10: Liver Cancer Cell Line Experiment

Minivectors encoding shFOXMI and Tp53 were tested using liver cancer cell lines (HepG2). The Tp53 that was used was a tetrameric p53. HepG2 cells were grown in completer media (Dulbecco's Modified Eagle Medium (DMEM), 10% fetal bovine serum (FBS), 1% Penicillin/Streptomycin). A 96-well cell culture plate was used and 7,000 cells were plated in each well (7 k cells/well). Each treatment was conducted in three wells (technical replicates).

The minivectors concentration for Tp53 and the relevant control Phosphoglycerate kinase (PGK)-NanoLuc minivector (i.e., a minivector that expresses NanoLuc—a bioluminescent protein) were each tested at 0.2 μg. For the minivector FOXM1 response was assessed testing at 0.2 μg and 0.4 μg along with its respective control, minivector shGFP (i.e., a minivector that knocks down Green Fluorescent Protein) testing at 0.2 μg and 0.4 μg.

Each of the minivectors were coated with Star-GalNac (Example 7) at a N/P ratio (amine groups of the star GalNac to phosphate groups of the minivector) of 10 via hotplate mixing in nitrogen at room temperature, forming the coated minivectors. Vortex mixing could also have been used.

Star-GalNac (Example 7) was administered as a control alone.

Cell treatment with each of the coated minivectors was conducted using media with BSA (bovine serum albumin) (DMEM, 0.1% BSA). After 24 hrs post-treatment, cells were harvested for MTT analysis. The used media was removed and 100 μL of fresh media was added. Then, 10 μL of MTT were added to each well and incubated for 4 hours at 37° C. in a CO₂ incubator. After this step, 100 μL of SDS-HCl solution was added to each well. This was followed by another 4 hours of incubation time at 37° C. in a CO₂ incubator. Thereafter, the samples were mixed by pipetting up and down and the absorbance was read at 570 nm using a plate reader. Relative cell growth (%) was calculated relative to the untreated cells (media only) as a background control.

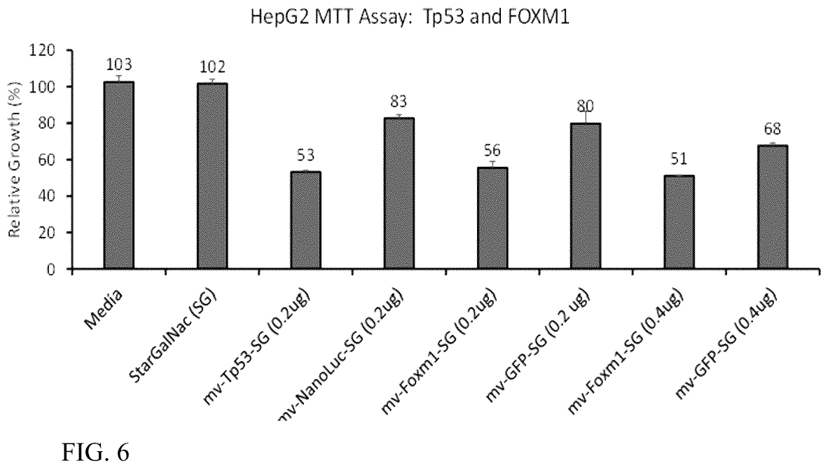

The achieved results as measured at 24 hours are shown in the chart of FIG. 6. In summary, GalNac-Star had a de minimis effect on cell growth. Tp53 and FOXM1 coated with GalNac-Star each exhibited a meaningful oncocidal benefit compared to the media of 103% (48.5% reduction of cancer cells for the lower-dose Tp53; 45.6% reduction of cancer cells for the lower-dose FOXM1; and 50.5% reduction for the cancer cells of the higher-dose FOXM1). GalNac-Star control minivectors reduced cell growth (19.4% reduction of cancer cells for the lower-dose NanoLuc; 22.3% reduction of cancer cells for the lower-dose GFP; and 34% reduction for the higher-dose GFP).

### Example 11: Administration to Mice (Prophetic Example)

Previous biodistribution assays relying on a Firefly luciferase approach published in Zhao (2018), suggest that polyplexes formed from an 8-arm Star polymer exhibit a strong lung tropism in healthy mice and that this tropism is independent of the first-pass organ encountered by the polyplex when delivered systemically (i.e., lung first-tail vein; liver first-intracardiac).

GalNac conjugated to a star polymer exhibiting a plurality of arms will exhibit a tropism for delivery to the liver when said polyplex is administered systemically. The liver tropism will happen regardless of the organ that the polyplex encounters first (e.g., lung or liver).

Healthy and diseased mice treated via Star-GalNac polyplexes will exhibit a strong liver tropism. To assess this liver tropism, DNA encoding for either Firefly luciferase or a Nanoluc fusion protein (e.g., NanoLuc-MSSmOrange—where said fusion protein improves the resolution of in vivo live imaging) will be employed. While the theoretical experiments refer to a NanoLuc-derivative, it is understood that similar results will result from the use of Firefly luciferase provided that experimental modifications specific to Firefly luciferase will be undertaken. Said modifications are broadly described in Zhao (2018).

While minivectors are specifically described, other nucleic acids could be optionally substituted including mRNA, siRNA, miRNA, saRNA, plasmid DNA, miniplasmids, nanoplasmids, recombinant minicircles, any closed-looped nucleic acid, synthetic circular DNA, synthetic DNA, “Doggybone” DNA, capped synthetic DNA, and others.

Mice will be intravenously injected with minivectors encoding for mv-NanoLucFusion alone or mv-NanoLuc coated with GalNac prepared at an N/P ratio of 10. Other N/P ratios are possible including 0.5, 1, 2, etc.

Bioluminescence imaging will be performed at certain time points after particle administration: 24 hours, 72 hours, 168 hours, and 336 hours. To image the NanoLuc activity, 5 μg (≈0.25 mg/kg) furimazine (≈40× dilution of Nano-Glo substrate) will be injected in 100 μl sterile PBS via tail vein and imaged mice on an IVIS Spectrum within 30 seconds of injection. Images will be taken with open filter and acquisition times from 1-60 seconds. The organs 24 h post administration will be collected and imaged in a 24-well plate immediately with an exposure time of 1 min. The images will be analyzed using Live Image 4.3 software. This in vivo bioluminescence imaging will be performed in the following 6 groups. For all of the 6 groups, the minivector (e.g., mv-CMV-NanoLuc alone; as control) will be administered or the minivector Nanoluc CMV promoter coated with Star-GalNac (mv-CMV-NanoLuc-Star-GalNac) will be administered. The following are the 6 different groups:
1) Minivector Biodistribution via IV ROA in healthy mice: healthy mice will receive either mv-CMV-NanoLuc alone or mv-CMV-NanoLuc-Star-GalNac through tail-vein injection (IV) without any anesthetic agents. This group will be the control group for the IV route of administration (“ROA”) to determine normal minivector biodistribution in healthy mice.
2) Minivector Biodistribution via IV ROA in mice with liver fibrosis: mice will be subjected to carbon tetrachloride (CCL4) to induce liver fibrosis and will receive either mv-CMV-NanoLuc alone or mv-CMV-NanoLuc-Star-GalNac through tail-vein injection (IV) without any anesthetic agents. This experimental group will be to determine the minivector biodistribution during liver fibrotic conditions.
3) Minivector Biodistribution via IV ROA in xenograft-mice with liver cancer cells: immunodeficient mice will be subjected to liver cancer cells (HepG2) to induce liver cancer and will receive either mv-CMV-NanoLuc alone or mv-CMV-NanoLuc-Star-GalNac through tail-vein injection (IV) without any anesthetic agents. This experimental group will be to determine the minivector biodistribution during liver cancer conditions.
4) Minivector Biodistribution via IC ROA in healthy mice: healthy mice will receive either mv-CMV-NanoLuc alone or mv-CMV-NanoLuc-Star-GalNac through intracardiac injection (IC). The mice will be injected IP with Ketamine and maintained, if necessary, with isoflurane. The animal will be laid dorsal side down on top of the sterile blue pad. The animal's front limbs will be taped down to the pad so that the chest is fully exposed. 70% ethanol will be rubbed gently on the chest to moisten the hair so that it lies down. With a 26 gauge needle and a 1 cc syringe loaded with DNA nanoparticles, the needle will be injected directly into the chest between the animal's sternum and third intercostals space, inserting the needle slowly into the second intercostals space. The intracardiac approach will be employed to confirm that the desired organ tropism (i.e., lung or liver) is observed independent of the initial organ contacted by the minivector (i.e., lung or liver) post-injection.
5) Minivector Biodistribution via IC ROA in mice with liver fibrosis: mice will be subjected to carbon tetrachloride (CCL4) to induce liver fibrosis and will receive either mv-CMV-NanoLuc alone or mv-CMV-NanoLuc-Star-GalNac through intracardiac injection (IC). This experimental group will be to determine the minivector biodistribution during liver fibrotic conditions.
6) Minivector Biodistribution via IC ROA in xenograft-mice with liver cancer cells: immunodeficient mice subjected to liver cancer cells (HepG2) to induce lung cancer will receive either mv-CMV-NanoLuc alone or mv-CMV-NanoLuc-Star-GalNac through intra-cardiac injection (IC). This experimental group will be to determine the minivector biodistribution during liver cancer conditions.

In the preceding experiments of Example 11, more than 15% of the administered Star-GalNac polyplexes will be delivered to organs (and not excreted) and will be delivered to the liver.

The present invention includes the following aspects/embodiments/features in any order and/or in any combination:
1. The present invention relates to conjugated composition comprising a nucleic acid sequence directly or indirectly conjugated to at least one N-Acetylgalactosamine (GalNAc) molecule or a derivative of a GalNAc molecule, by a linker, wherein the nucleic acid sequence is closed-looped, and the conjugated composition is structured to enable the at least one GalNAc molecule to deliver the nucleic acid sequence into a cell.
2. The conjugated composition or method or other embodiment of any preceding or following embodiment/feature/aspect, wherein the cell is a liver cell.
3. The conjugated composition or method or other embodiment of any preceding or following embodiment/feature/aspect, wherein the cell is a damaged cell, an abnormal cell, or a diseased cell.
4. The conjugated composition or method or other embodiment of any preceding or following embodiment/feature/aspect, wherein the nucleic acid sequence is a supercoiled DNA minicircle having from 100 base pairs to 5 kilo base pairs.
5. The conjugated composition or method or other embodiment of any preceding or following embodiment/feature/aspect, wherein the supercoiled DNA minicircle consists of a promoter and a therapeutic sequence for treatment of the cell.
6. The conjugated composition or method or other embodiment of any preceding or following embodiment/feature/aspect, wherein the supercoiled DNA minicircle is absent of a selection marker and a bacterial origin of replication.
7. The conjugated composition or method or other embodiment of any preceding or following embodiment/feature/aspect, wherein the supercoiled DNA minicircle has from 250 base pairs to 3.5 kilo base pairs.
8. The conjugated composition or method or other embodiment of any preceding or following embodiment/feature/aspect, wherein the supercoiled DNA minicircle has from 500 base pairs to 2.5 kilo base pairs.
9. The conjugated composition or method or other embodiment of any preceding or following embodiment/feature/aspect, wherein the supercoiled DNA minicircle has an outer width of from 2 nm to 80 nm.
10. The conjugated composition or method or other embodiment of any preceding or following embodiment/feature/aspect, wherein the supercoiled DNA minicircle has a length of from 20 nm to 300 nm.
11. The conjugated composition or method or other embodiment of any preceding or following embodiment/feature/aspect, wherein the linker is a linear molecule.
12. The conjugated composition or method or other embodiment of any preceding or following embodiment/feature/aspect, wherein the linker is selected from the group consisting of hydrocarbon chains, ethers, esters, and a combination thereof.
13. The conjugated composition or method or other embodiment of any preceding or following embodiment/feature/aspect, wherein the nucleic acid sequence is indirectly conjugated to the at least one GalNAc molecule by a cationic or neutrally charged polymer and is in the form of an encapsulated nucleic acid particle.
14. The conjugated composition or method or other embodiment of any preceding or following embodiment/feature/aspect, further comprising at least one group bonded to at least one of the cationic or neutrally charged polymer and the nucleic acid sequence, wherein the at least one group actively or passively targets diseased cells.
15. The conjugated composition or method or other embodiment of any preceding or following embodiment/feature/aspect, wherein the encapsulated nucleic acid particle has a length of from 20 nm to 250 nm.
16. The conjugated composition or method or other embodiment of any preceding or following embodiment/feature/aspect, wherein the cationic or neutrally charged polymer comprises a plurality of polymeric arms.
17. The conjugated composition or method or other embodiment of any preceding or following embodiment/feature/aspect, wherein the plurality of polymeric arms comprises at least eight polymeric arms.
18. The conjugated composition or method or other embodiment of any preceding or following embodiment/feature/aspect, wherein each of the plurality of polymer arms comprises a biodegradable polypeptide, a polysaccharide, or a combination thereof.
19. The conjugated composition or method or other embodiment of any preceding or following embodiment/feature/aspect, wherein each of the plurality of arms comprises a first side comprising an amine that bonds with the nucleic acid sequence, a second side comprising a respective linker that bonds to a respective at least one GalNAc molecule, one of the respective linkers is the linker, and one of the respective at least one GalNAc molecules is the at least one GalNAc molecule.
20. The conjugated composition or method or other embodiment of any preceding or following embodiment/feature/aspect, wherein the at least one GalNAc molecule comprises at least two GalNAc molecules.
21. The conjugated composition or method or other embodiment of any preceding or following embodiment/feature/aspect, wherein the at least one GalNAc molecule comprises at least three GalNAc molecules.
22. The conjugated composition or method or other embodiment of any preceding or following embodiment/feature/aspect, wherein the nucleic acid sequence is directly conjugated to the at least one GalNAc molecule by the linker.
23. The conjugated composition or method or other embodiment of any preceding or following embodiment/feature/aspect, wherein the at least one GalNAc molecule is bonded to an oligonucleotide by the linker, and the oligonucleotide comprises a counterpart sequence that bonds with an exposed segment of the nucleic acid sequence.
24. The conjugated composition or method or other embodiment of any preceding or following embodiment/feature/aspect, wherein the exposed segment of the nucleic acid sequence bonded to the oligonucleotide is a local triplex.
25. The conjugated composition or method or other embodiment of any preceding or following embodiment/feature/aspect, wherein the oligonucleotide has less than 30 bases.
26. The conjugated composition or method or other embodiment of any preceding or following embodiment/feature/aspect, wherein the nucleic acid sequence comprises an exposed phosphate group, the at least one GalNAc molecule is directly bonded to the exposed phosphate group by the linker.
27. The conjugated composition or method or other embodiment of any preceding or following embodiment/feature/aspect, wherein the at least one GalNAc molecule comprises at least two GalNAc molecules.
28. The conjugated composition or method or other embodiment of any preceding or following embodiment/feature/aspect, wherein the at least one GalNAc molecule comprises at least three GalNAc molecules.
29. The conjugated composition or method or other embodiment of any preceding or following embodiment/feature/aspect, wherein the nucleic acid sequence is underwound such that it has flipped out bases, and the at least one GalNAc molecule is bonded to at least one of the flipped-out bases by the linker.
30. The conjugated composition or method or other embodiment of any preceding or following embodiment/feature/aspect, wherein an end of the nucleic acid sequence is nicked with a sequence-specific endonucleic nickase, and the at least one GalNAc molecule is bonded to the end of the nucleic acid sequence by the linker.
31. The conjugated composition or method or other embodiment of any preceding or following embodiment/feature/aspect, wherein the nucleic acid sequence comprises at least two nicking sites at which a segment of the nucleic acid sequence is unhybridized such that it has a single stranded gap, the at least one GalNAc molecule is bonded to a single oligonucleotide by the linker, and the single oligonucleotide is bonded to the nucleic acid sequence within the single stranded gap.
32. An embodiment that is a method comprising: contacting a living cell with a conjugated composition, the conjugated composition comprising a therapeutic supercoiled DNA minicircle having from 100 base pairs to 5 kilo base pairs, the therapeutic supercoiled DNA minicircle directly or indirectly conjugated to at least one N-Acetylgalactosamine (GalNAc) molecule or a derivative of a GalNAc molecule, by a linker, wherein the at least one GalNAc molecule of the conjugated composition binds to a receptor of the cell, the conjugated composition is taken up in an endosome wherein the conjugated composition dissociates from the receptor, the at least one GalNAc molecule is cleaved from the therapeutic supercoiled DNA minicircle at the linker, and the therapeutic supercoiled DNA minicircle metabolizes in the living cell to form a treated cell.
33. The method or conjugated composition or other embodiment of any preceding or following embodiment/feature/aspect, wherein the living cell is a liver cell.
34. The method or conjugated composition or other embodiment of any preceding or following embodiment/feature/aspect, further comprising administering the conjugated composition to a human or other animal.
35. An embodiment that is, in combination, a treated cell and a remnant N-Acetylgalactosamine (GalNAc) molecule comprising remnants of a linker, wherein: the treated cell comprises a human liver cell and a therapeutic supercoiled DNA minicircle metabolizing within the human liver cell, the therapeutic supercoiled DNA minicircle having been formed from cleavage of a conjugated composition, the conjugated composition comprising the therapeutic supercoiled DNA minicircle directly or indirectly conjugated to a GalNAc molecule, by the linker; and the remnant GalNAc molecule is the GalNAc molecule of the conjugated composition, having a remnant of the linker attached thereto.

The present invention can include any combination of these various features or embodiments above and/or below as set forth in sentences and/or paragraphs. Any combination of disclosed features herein is considered part of the present invention and no limitation is intended with respect to combinable features.

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. Herein “about” means within a standard deviation of plus or minus 5 percent, where applicable. It is not intended that the scope of the invention be limited to the specific values recited when defining a range.

Other embodiments of the present invention will be apparent to those skilled in the art from consideration of the present specification and practice of the present invention disclosed herein. It is intended that the present specification and examples be considered as exemplary only with a true scope and spirit of the invention being indicated by the following claims and equivalents thereof.

What is claimed is:

1. A conjugated composition comprising a nucleic acid sequence directly or indirectly conjugated to at least one N-Acetylgalactosamine (GalNAc) molecule or a derivative of a GalNAc molecule, by a linker, wherein the nucleic acid sequence is closed-looped, and the conjugated composition is structured to enable the at least one GalNAc molecule to deliver the nucleic acid sequence into a cell.

2. The conjugated composition of claim 1, wherein the cell is a liver cell.

3. The conjugated composition of claim 1, wherein the cell is a damaged cell, an abnormal cell, or a diseased cell.

4. The conjugated composition of claim 1, wherein the nucleic acid sequence is a supercoiled DNA minicircle having from 100 base pairs to 5 kilo base pairs.

5. The conjugated composition of claim 4, wherein the supercoiled DNA minicircle consists of a promoter and a therapeutic sequence for treatment of the cell.

6. The conjugated composition of claim 4, wherein the supercoiled DNA minicircle is absent of a selection marker and a bacterial origin of replication.

7. The conjugated composition of claim 4, wherein the supercoiled DNA minicircle has from 250 base pairs to 3.5 kilo base pairs.

8. The conjugated composition of claim 4, wherein the supercoiled DNA minicircle has from 500 base pairs to 2.5 kilo base pairs.

9. The conjugated composition of claim 4, wherein the supercoiled DNA minicircle has a outer width of from 2 nm to 80 nm.

10. The conjugated composition of claim 4, wherein the supercoiled DNA minicircle has a length of from 20 nm to 300 nm.

11. The conjugated composition of claim 1, wherein the linker is a linear molecule.

12. The conjugated composition of claim 11, wherein the linker is selected from the group consisting of hydrocarbon chains, ethers, esters, and a combination thereof.

13. The conjugated composition of claim 1, wherein the nucleic acid sequence is indirectly conjugated to the at least one GalNAc molecule by a cationic or neutrally charged polymer and is in the form of an encapsulated nucleic acid particle.

14. The conjugated composition of claim 13, further comprising at least one group bonded to at least one of the cationic or neutrally charged polymer and the nucleic acid sequence, wherein the at least one group actively or passively targets diseased cells.

15. The conjugated composition of claim 13, wherein the encapsulated nucleic acid particle has a length of from 20 nm to 250 nm.

16. The conjugated composition of claim 13, wherein the cationic or neutrally charged polymer comprises a plurality of polymeric arms.

17. The conjugated composition of claim 16, wherein the plurality of polymeric arms comprises at least eight polymeric arms.

18. The conjugated composition of claim 16, wherein each of the plurality of polymer arms comprises a biodegradable polypeptide, a polysaccharide, or a combination thereof.

19. The conjugated composition of claim 16, wherein each of the plurality of arms comprises a first side comprising an amine that bonds with the nucleic acid sequence, a second side comprising a respective linker that bonds to a respective at least one GalNAc molecule, one of the respective linkers is the linker, and one of the respective at least one GalNAc molecules is the at least one GalNAc molecule.

20. The conjugated composition of claim 19, wherein the at least one GalNAc molecule comprises at least two GalNAc molecules.

21. The conjugated composition of claim 19, wherein the at least one GalNAc molecule comprises at least three GalNAc molecules.

22. The conjugated composition of claim 1, wherein the nucleic acid sequence is directly conjugated to the at least one GalNAc molecule by the linker.

23. The conjugated composition of claim 22, wherein the at least one GalNAc molecule is bonded to an oligonucleotide by the linker, and the oligonucleotide comprises a counterpart sequence that bonds with an exposed segment of the nucleic acid sequence.

24. The conjugated composition of claim 23, wherein the exposed segment of the nucleic acid sequence bonded to the oligonucleotide is a local triplex.

25. The conjugated composition of claim 24, wherein the oligonucleotide has less than 30 bases.

26. The conjugated composition of claim 22, wherein the nucleic acid sequence comprises an exposed phosphate group, the at least one GalNAc molecule is directly bonded to the exposed phosphate group by the linker.

27. The conjugated composition of claim 26, wherein the at least one GalNAc molecule comprises at least two GalNAc molecules.

28. The conjugated composition of claim 26, wherein the at least one GalNAc molecule comprises at least three GalNAc molecules.

29. The conjugated composition of claim 22, wherein the nucleic acid sequence is underwound such that it has flipped out bases, and the at least one GalNAc molecule is bonded to at least one of the flipped-out bases by the linker.

30. The conjugated composition of claim 22, wherein an end of the nucleic acid sequence is nicked with a sequence-specific endonucleic nickase, and the at least one GalNAc molecule is bonded to the end of the nucleic acid sequence by the linker.

31. The conjugated composition of claim 22, wherein the nucleic acid sequence comprises at least two nicking sites at which a segment of the nucleic acid sequence is unhybridized such that it has a single stranded gap, the at least one GalNAc molecule is bonded to a single oligonucleotide by the linker, and the single oligonucleotide is bonded to the nucleic acid sequence within the single stranded gap.

32. A method comprising:
contacting a living cell with a conjugated composition, the conjugated composition comprising a therapeutic supercoiled DNA minicircle having from 100 base pairs to 5 kilo base pairs, the therapeutic supercoiled DNA minicircle directly or indirectly conjugated to at least one N-Acetylgalactosamine (GalNAc) molecule or a derivative of a GalNAc molecule, by a linker, wherein
the at least one GalNAc molecule of the conjugated composition binds to a receptor of the cell,
the conjugated composition is taken up in an endosome wherein the conjugated composition dissociates from the receptor,
the at least one GalNAc molecule is cleaved from the therapeutic supercoiled DNA minicircle at the linker, and
the therapeutic supercoiled DNA minicircle metabolizes in the living cell to form a treated cell.

33. The method of claim 32, wherein the living cell is a liver cell.

34. The method of claim 32, further comprising administering the conjugated composition to a human or other animal.

35. In combination, a treated cell and a remnant N-Acetylgalactosamine (GalNAc) molecule comprising remnants of a linker, wherein:
the treated cell comprises a human liver cell and a therapeutic supercoiled DNA minicircle metabolizing within the human liver cell, the therapeutic supercoiled DNA minicircle having been formed from cleavage of a conjugated composition, the conjugated composition comprising the therapeutic supercoiled DNA minicircle directly or indirectly conjugated to a GalNAc molecule, by the linker; and
the remnant GalNAc molecule is the GalNAc molecule of the conjugated composition, having a remnant of the linker attached thereto.
